(19) United States
(12) Patent Application Publication
(10) Pub. No.: US 2013/0171208 A1
(43) Pub. Date: Jul. 4, 2013

(54) COMPOSITIONS OF NANOPARTICLES AND METHODS OF MAKING THE SAME

(71) Applicant: Jeffrey W. Smith, San Diego, CA (US)

(72) Inventors: Jeffrey W. Smith, San Diego, CA (US); Hui Xie, San Diego, CA (US)

(73) Assignee: Jeffrey W Smith, San Diego, CA (US)

(21) Appl. No.: 13/779,022

(22) Filed: Feb. 27, 2013

Related U.S. Application Data
(63) Continuation of application No. 12/908,775, filed on Oct. 20, 2010, now abandoned.
(60) Provisional application No. 61/253,814, filed on Oct. 21, 2009.
(60) Provisional application No. 61/355,120, filed on Jun. 15, 2010.
(60) Provisional application No. 61/356,450, filed on Jun. 18, 2010.

(51) Int. Cl. A61K 47/30

(52) U.S. Cl. CPC A61K 47/30
USPC 424/400; 422/129; 422/187; 422/119; 422/240; 514/772.3

(57) ABSTRACT

Disclosed herein are compositions of nanoparticles. In some embodiments, the nanoparticles are Janus particles, where each particle includes a first component and second component that are exposed to the surface of the particle. Also, disclosed are methods and systems for making a composition of nanoparticles. Finally, a method of treating a mammal by administering a composition of nanoparticles is disclosed.

### CROSS-REFERENCE TO RELATED APPLICATIONS

This application claims the benefit of priority to U.S. Provisional Application No. 61/253,814, filed Oct. 21, 2009; U.S. Provisional Application No. 61/355,120, filed Jun. 15, 2010; and U.S. Provisional Application No. 61/356,450, filed Jun. 18; 2010. The contents of each of the priority documents are hereby incorporated by reference in their entirety.

### BACKGROUND

1. Field

The present application relates to compositions of nanoparticles and to methods for preparing compositions of nanoparticles that can be used in the fields of chemistry and medicine.

2. Description

Particulate drug delivery systems can be developed for delivering drugs to a subject. However, in order to effectively use particulate drug delivery systems, the particle characteristics (e.g., size, composition, etc.) may require precise control to obtain, for example, targeted delivery to a desired tissue or cell. Unfortunately, current methods for manufacturing particulate drug delivery systems provide limited control over particle characteristics. For example, it may be difficult to control the particle diameter, particularly at the nanometer scale.

Particulate systems generally may also be used in other fields. For example, particles may be used to improve the properties of various adhesives or coatings.

Particles with two compartments, and distinct surfaces, are called Janus particles after the mythological Roman god of gates, who is typically shown with two faces peering in opposite directions. Most Janus particles are spherically shaped, and thus have two discernible hemi-spheres, but cylinders and discs have also been developed. For a review of Janus particles, see Walther, A.; Muller, A., Soft Matter, 2008, Vol. 4, pg. 663-668, which is hereby incorporated by reference in its entirety. Because of their dimorphic nature, Janus particles provide the opportunity for applications not possible with particles having a homogeneous surface. Such applications include electronically controlled display panels, emulsifiers, optically modulated nanosensors, self-propelled nano-vehicles, and self-assembly of interesting superstructures.

### SUMMARY

Some embodiments includes a method of making Janus particles comprising: (a) providing at least a first liquid feed stream and a second liquid feed stream; and (b) intermixing the first liquid feed stream and the second liquid feed stream with a dispersing stream, thereby solidifying components of the first liquid feed stream and the second liquid feed stream into a plurality of Janus particles dispersed in the dispersing stream.

Some embodiments includes a method of making Janus particles comprising: (a) providing at least a first liquid feed stream and a second liquid feed stream; and (b) intermixing the first liquid feed stream and the second liquid feed stream with a dispersing stream, thereby solidifying components of the first liquid feed stream and the second liquid feed stream into a plurality of Janus particles dispersed in the dispersing stream, wherein: the first liquid feed stream comprises a first polymer and the second liquid feed stream comprises a second component that is substantially different from the first polymer; and at least a portion of the Janus particles comprise the first polymer and the second component.

In some embodiments, a portion of the first liquid feed stream contacts a portion of the second liquid feed stream before the portion of the first liquid feed stream and/or the portion of the second liquid feed stream contacts the dispersing stream.

In some embodiments, wherein a portion of the first liquid feed stream, a portion of the second liquid feed stream and the dispersing stream all initially contact each other at about the time.

In some embodiments, wherein a portion of the first liquid feed stream and/or a portion of the second liquid feed stream contacts the dispersing stream before the portion of the first liquid feed stream contacts the portion of the second liquid feed stream.

In some embodiments, the first liquid feed stream further comprises a first solvent that is at least partially miscible in the dispersing stream. In some embodiments, the first liquid feed stream further comprises a first solvent selected from the group consisting of 1,4 dioxane, tetrahydrofuran (THF), acetone, acetonitrile, dimethyl sulfoxide (DMSO), dimethylformamide (DMF), acids, and C1-C8 alcohols.

In some embodiments, the second liquid feed stream further comprises a second solvent that is at least partially miscible in the dispersing stream. In some embodiments, the second liquid feed stream further comprises a second solvent selected from the group consisting of 1,4 dioxane, tetrahydrofuran (THF), acetone, acetonitrile, dimethyl sulfoxide (DMSO), dimethylformamide (DMF), acids, and C₁-C₈ alcohols.

In some embodiments, the first liquid feed stream and the second liquid feed stream are configured to solidify the components of the first liquid feed stream and the second liquid feed stream into the plurality of Janus particles before substantial intermixing of the first polymer and the second component.

In some embodiments, the first liquid feed stream has a first diameter in the range of about 1 μm to about 1 mm and the second liquid feed stream has a second diameter in the range of about 1 μm to about 1 mm.

In some embodiments, the dispersing stream has a third diameter that is at least 2 times larger than the first diameter and the second diameter. In some embodiments, the dispersing stream has a third diameter that is at least 5 times larger than the first diameter and the second diameter.

In some embodiments, the plurality of Janus particles has an average diameter in the range of about 10 nm to about 10 μm.

In some embodiments, the first liquid feed stream has a first flow rate in the range of about 1 μL/hr. to about 100 mL/min. and the second liquid feed stream has a second flow rate in the range of about 1 μL/hr. to about 100 mL/min.

In some embodiments, the dispersing feed stream has a third flow rate that is in the range of about 2 times greater to about 10 times greater than the first feed stream. In some embodiments, the dispersing feed stream has a third flow rate that is in the range of about 3 times greater to about 6 times greater than the first feed stream.

In some embodiments, the first liquid feed stream and the dispersing stream intersect at an angle θ₁ that is in the range of about 5 degrees to about 175 degrees. In some embodiments, the first liquid feed stream and the dispersing stream intersect at an angle θ₁ that is in the range of about 0 degrees to about 170 degrees. In some embodiments, the first liquid feed stream and the dispersing stream intersect at an angle θ₁ that is in the range of about 10 degrees to about 180 degrees. In some embodiments, the first liquid feed stream and the dispersing stream intersect at an angle θ1 that is about 0 degrees. In some embodiments, the first liquid feed stream and the dispersing stream intersect at an angle θ1 that is about 90 degrees. In some embodiments, the second feed stream and the dispersing stream intersect at an angle θ2 that is in the range of about 5 degrees to about 175 degrees.

In some embodiments, the second feed stream and the dispersing stream intersect at an angle θ2 that is in the range of about 10 degrees to about 180 degrees. In some embodiments, the second feed stream and the dispersing stream intersect at an angle θ₂ that is about 0 degrees. In some embodiments, the second feed stream and the dispersing stream intersect at an angle θ2 that is about 90 degrees.

In some embodiments, the first liquid feed stream has a first outlet having a first center; the second liquid feed stream has a second outlet having a second center; and the dispersing stream and a vector from the first center to the second center intersect at an angle ψ that is in the range of about 5 degrees to about 355 degrees. In some embodiments, the first liquid feed stream has a first outlet having a first center; the second liquid feed stream has a second outlet having a second center; and the dispersing stream and a vector from the first center to the second center intersect at an angle ψ that is in the range of about −175 degrees to about 175 degrees.

In some embodiments, the temperature of the dispersing stream is at least 1° C. lower than the temperature of at least one of the first liquid feed stream and the second liquid feed stream. In some embodiments, the temperature of the dispersing stream is at least 5° C. lower than the temperature of at least one of the first liquid feed stream and the second liquid feed stream. In some embodiments, the temperature of the dispersing stream is at least 10° C. lower than the temperature of at least one of the first liquid feed stream and the second liquid feed stream. In some embodiments, the temperature of the dispersing stream is at least 25° C. lower than the temperature of at least one of the first liquid feed stream and the second liquid feed stream.

In some embodiments, the first polymer is miscible in the first feed stream and substantially immiscible in the dispersing stream; and the second component is miscible in the second feed stream and substantially immiscible in the dispersing stream.

In some embodiments, at least about 1 g/L of the first polymer is dispersed in the first feed stream and at least about 1 g/L of the second component is dispersed in the second feed stream.

In some embodiments, the viscosity of the first liquid feed stream is configured so that the first liquid feed stream flows at a rate of at least 1 μL/hr. when a pressure of no more than 7 MPa is applied. In some embodiments, the viscosity of the second liquid feed stream is configured so that the second liquid feed stream flows at a rate of at least 1 μL/hr. when a pressure of no more than 7 MPa is applied. In some embodiments, the viscosity of the dispersing stream is configured so that the dispersing stream flows at a rate of at least 2 μL/hr. when a pressure of no more than 7 MPa is applied.

In some embodiments, the method further comprises applying an energy source to the plurality of Janus particles dispersed in the dispersing stream for a time that is effective to modify said plurality of Janus particles.

In some embodiments, the method further comprises recycling a portion of the dispersing stream after intermixing with the first feed stream and second feed stream.

In some embodiments, the first liquid feed stream comprises a first pharmaceutical agent. In some embodiments, the second liquid feed stream comprises a second pharmaceutical agent. In some embodiments, the first pharmaceutical agent is the same as the second pharmaceutical agent. In some embodiments, the first pharmaceutical agent is different than the second pharmaceutical agent.

In some embodiments, the first pharmaceutical agent has a first partition coefficient, the second pharmaceutical agent has a second partition coefficient, and a difference between the first partition coefficient and the second partition coefficient is at least about 1. In some embodiments, the difference between the first partition coefficient and the second partition coefficient is at least about 1.5. In some embodiments, the difference between the first partition coefficient and the second partition coefficient is at least about 2. In some embodiments, the first partition coefficient is at least about 2.5 and the second partition coefficient is no more than about 2.5.

Some embodiments include a composition comprising a plurality of Janus particles, each Janus particle comprising a first component and a second component that is substantially different from the first component, wherein: the Janus particles have an average size in the range of about 10 nm to about 10000 nm; and at least part of the first component and at least part of the second component are exposed at an outer surface of the Janus particle.

Some embodiments include a composition comprising a plurality of Janus particles, each Janus particle comprising: a first component comprising a first polymer; and a second component that is substantially different from the first component, wherein: the Janus particles have an average size in the range of about 10 nm to about 2000 nm; and at least part of the first component and at least part of the second component are exposed at an outer surface of the Janus particle.

In some embodiments, the first polymer comprises a recurring unit of the formula (I):

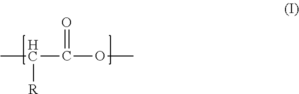

wherein R is selected from hydrogen and methyl.

In some embodiments, the first polymer is poly(lactide-co-glycolide (PLGA) or a PLGA-based copolymer. In some embodiments, the first polymer is selected from the group consisting of polyethylene glycol (PEG), poly(lactic acid-co-glycolic acid) (PLGA), copolymers of PLGA and PEG, copolymers of poly(lactide-co-glycolide) and PEG, polyglycolic acid (PGA), copolymers of PGA and PEG, poly-L-lactic acid (PLLA), copolymers of PLLA and PEG, poly-D-lactic acid (PDLA), copolymers of PDLA and PEG, poly-D,L-lactic acid (PDLLA), copolymers of PDLLA and PEG, poly(ortho ester), copolymers of poly(ortho ester) and PEG, poly(caprolactone), copolymers of poly(caprolactone) and PEG, polylysine, copolymers of polylysine and PEG, polyethylene imine, copolymers of polyethylene imine and PEG, polyhydroxyacids, polyanhydrides, polyhydroxyalkanoates, poly(L-lactide-co-L-lysine), poly(serine ester), poly(4-hydroxy-L-proline ester), poly[α-(4-aminobutyl)-L-glycolic acid, derivatives thereof, combinations thereof and copolymers thereof.

In some embodiments, the second component comprises an ingredient selected from the group consisting of a pharmaceutical agent, a biomedical imaging agent and a second polymer.

In some embodiments, at least a portion of the Janus particles further comprise one or more additional components that are different from the first component and the second component.

In some embodiments, the one or more additional components comprises a second ingredient selected from the group consisting of a pharmaceutical agent, a biomedical imaging agent and a polymer.

In some embodiments, the first component is a solid. In some embodiments, the second component is a solid. In some embodiments, the one or more additional components are a solid.

In some embodiments, at least a portion of the Janus particles comprise at least about 30% of the first component by weight. In some embodiments, at least a portion of the Janus particles comprise at least about 70% of the first component by weight. In some embodiments, at least a portion of the Janus particles comprise at least about 90% of the first component by weight.

In some embodiments, at least a portion of the Janus particles comprise no more than about 99.5% of the first component by weight. In some embodiments, at least a portion of the Janus particles comprise no more than about 95% of the first component by weight. In some embodiments, at least a portion of the Janus particles comprise no more than about 80% of the first component by weight. In some embodiments, at least a portion of the Janus particles comprise at least about 0.5% of the second component by weight.

In some embodiments, at least a portion of the Janus particles comprise at least about 5% of the second component by weight. In some embodiments, at least a portion of the Janus particles comprise at least about 10% of the second component by weight. In some embodiments, at least a portion of the Janus particles comprise at least about 50% of the second component by weight.

In some embodiments, at least a portion of the Janus particles comprise no more than about 20% of the second component by weight. In some embodiments, at least a portion of the Janus particles comprise no more than about 15% of the second component by weight. In some embodiments, at least a portion of the Janus particles comprise no more than about 5% of the second component by weight.

In some embodiments, the Janus particles have two distinct phases.

In some embodiments, the composition comprises at least 1 ppm Janus particles by weight. In some embodiments, the composition has a mass of at least 100 mg.

In some embodiments, the first component comprises a first pharmaceutical agent. In some embodiments, the second component comprises a second pharmaceutical agent. In some embodiments, the first pharmaceutical agent is the same as the second pharmaceutical agent. In some embodiments, the first pharmaceutical agent is different than the second pharmaceutical agent.

In some embodiments, the first pharmaceutical agent has a first partition coefficient, the second pharmaceutical agent has a second partition coefficient, and a difference between the first partition coefficient and the second partition coefficient is at least about 1. In some embodiments, the difference between the first partition coefficient and the second partition coefficient is at least about 1.5. In some embodiments, the difference between the first partition coefficient and the second partition coefficient is at least about 2. In some embodiments, the first partition coefficient is at least about 2.5 and the second partition coefficient is no more than about 2.5.

Some embodiments include a system for making a plurality of Janus particles, comprising: a first feed channel; a second feed channel; and a dispersing channel, wherein: the first feed channel has a first outlet that is operably connected to the dispersing channel; the second feed channel has a second outlet that is operably connected to the dispersing channel; the first outlet and the second outlet are no more than about 5 mm apart; and the first outlet and the second outlet are within about 1 mm of the dispersing channel; and the first feed channel has a first diameter in the range of about 10 μm to about 1 mm; the second feed channel has a second diameter in the range of about 10 μm to about 1 mm; and the dispersing channel has a third diameter that is at least 2 times larger than the first diameter.

In some embodiments, the system further comprises one or more pumps configured to displace a substance in the first feed channel, the second feed channel and/or the dispersing channel

In some embodiments, the system further comprises a means for isolating Janus particles dispersed in the dispersing channel, the isolating means being operably connected to the dispersing channel. In some embodiments, the isolating means comprises a filter.

In some embodiments, the dispersing channel forms a closed loop.

In some embodiments, the system further comprises a processor in communication with one or more pumps and/or one or more measuring devices.

In some embodiments, the system further comprises one or more additional feed channels connected to the dispersion channel at a common intersection with any other feed channel.

In some embodiments, the first outlet and the second outlet are operably connected to a cojoining chamber that is operably connected to the dispersing channel. In some embodiments, the cojoining chamber is configured so that the first outlet or the second outlet is at least about 10 nm from the dispersing channel. In some embodiments, the cojoining chamber is configured so that at least one of the first outlet and the second outlet is no more than about 100 μm from the dispersing channel.

In some embodiments, at least one of the first feed channel, the second feed channel and the dispersing channel is prepared by lithography, embossing, or molding of a polymer.

In some embodiments, at least one of the first feed channel, the second feed channel and the dispersing channel is a plastic tubing or a stainless steel tubing.

Some embodiments include a method of treating a mammal comprising administering to said mammal a pharmaceutically effective amount of a composition that comprises a plurality of Janus particles, wherein the plurality of Janus particles comprises: a first component comprising a first pharmaceutical agent; and a second component that is substantially different from the first component, wherein: the plurality of Janus particles have an average size in the range of about 10 nm to about 2000 nm; and at least part of the first component and at least part of the second component are exposed at an outer surface of the Janus particles.

In some embodiments, the second component comprises a second pharmaceutical agent. In some embodiments, the first pharmaceutical agent is the same as the second pharmaceutical agent. In some embodiments, the first pharmaceutical agent is different than the second pharmaceutical agent.

In some embodiments, the first pharmaceutical agent has a first partition coefficient, the second pharmaceutical agent has a second partition coefficient, and a difference between the first partition coefficient and the second partition coefficient is at least about 1. In some embodiments, the difference between the first partition coefficient and the second partition coefficient is at least about 1.5. In some embodiments, the difference between the first partition coefficient and the second partition coefficient is at least about 2. In some embodiments, the first partition coefficient is at least about 2.5 and the second partition coefficient is no more than about 2.5.

Some embodiments disclosed herein include a method of making nanoparticles, comprising: providing a liquid feed stream; intermixing the liquid feed stream with a dispersing stream, thereby solidifying components of the liquid feed stream into a plurality of nanoparticles dispersed in the dispersing stream, wherein: the dispersing stream has a diameter greater than about 500 μm; and at least 20% of said plurality of nanoparticles have a first diameter that is no more than about 1/200 of the diameter of the liquid feed stream.

In some embodiments, at least 40% of said plurality of nanoparticles have said first diameter. In some embodiments, at least 50% of said plurality of nanoparticles have said first diameter. In some embodiments, at least 60% of said plurality of nanoparticles have said first diameter. In some embodiments, at least 70% of said plurality of nanoparticles have said first diameter. In some embodiments, at least 80% of said plurality of nanoparticles have said first diameter. In some embodiments, at least 90% of said plurality of nanoparticles have said first diameter. In some embodiments, at least 95% of said plurality of nanoparticles have said first diameter.

In some embodiments, the first diameter is no more than about 1/400 of the diameter of the liquid feed stream. In some embodiments, the first diameter is no more than about 1/500 of the diameter of the liquid feed stream. In some embodiments, the first diameter is no more than about 1/1000 of the diameter of the liquid feed stream.

In some embodiments, the first diameter is no more than about 1000 nm. In some embodiments, the first diameter is no more than about 500 nm. In some embodiments, the first diameter is no more than about 300 nm. In some embodiments, the first diameter is no more than about 250 nm In some embodiments, the first diameter is no more than about 200 nm.

In some embodiments, the first diameter is at least about 10 nm. In some embodiments, the first diameter is at least about 20 nm. In some embodiments, the first diameter is at least about 50 nm. In some embodiments, the first diameter is at least about 100 nm. In some embodiments, the first diameter is at least about 200 nm.

In some embodiments, the liquid feed stream further comprises a first solvent that is at least partially miscible in the dispersing stream.

In some embodiments, the liquid feed stream further comprises a first solvent selected from the group consisting of 1,4 dioxane, tetrahydrofuran (THF), acetone, acetonitrile, dimethyl sulfoxide (DMSO), dimethylformamide (DMF), acids, and C1-C8 alcohols.

In some embodiments, the liquid feed stream comprises a polymer.

In some embodiments, the polymer is selected from the group consisting of polyethylene glycol (PEG), poly(lactic acid-co-glycolic acid) (PLGA), copolymers of PLGA and PEG, copolymers of poly(lactide-co-glycolide) and PEG, polyglycolic acid (PGA), copolymers of PGA and PEG, poly-L-lactic acid (PLLA), copolymers of PLLA and PEG, poly-D-lactic acid (PDLA), copolymers of PDLA and PEG, poly-D,L-lactic acid (PDLLA), copolymers of PDLLA and PEG, poly(ortho ester), copolymers of poly(ortho ester) and PEG, poly(caprolactone), copolymers of poly(caprolactone) and PEG, polylysine, copolymers of polylysine and PEG, polyethylene imine, copolymers of polyethylene imine and PEG, polyhydroxyacids, polyanhydrides, polyhydroxyalkanoates, poly(L-lactide-co-L-lysine), poly(serine ester), poly(4-hydroxy-L-proline ester), poly[α-(4-aminobutyl)-L-glycolic acid, derivatives thereof, combinations thereof and copolymers thereof.

In some embodiments, the diameter of the dispersing stream is at least about 1000 In some embodiments, the diameter of the dispersing stream is at least about 2000 In some embodiments, the diameter of the dispersing stream is at least about 5000 μm.

In some embodiments, the diameter of the dispersing stream is no more than about 10000 μm. In some embodiments, the diameter of the dispersing stream is no more than about 7500 μm. In some embodiments, the diameter of the dispersing stream is no more than about 5000 μm. In some embodiments, the diameter of the dispersing stream is no more than about 2000 μm.

In some embodiments, the liquid feed stream has a flow rate in the range of about 1 μL/hr to about 100 mL/min. In some embodiments, the dispersing stream has a flow rate of at least about 10 mL/min. In some embodiments, the dispersing stream has a flow rate of at least about 20 mL/min. In some embodiments, the dispersing stream has a flow rate of at least about 40 mL/min.

In some embodiments, the liquid feed stream further comprises a first solvent that is at least partially miscible in the dispersing stream.

In some embodiments, the temperature of the dispersing stream is at least 1° C. lower than the temperature of the liquid feed stream. In some embodiments, the temperature of the dispersing stream is at least 5° C. lower than the temperature of the liquid feed stream. In some embodiments, the temperature of the dispersing stream is at least 10° C. lower than the temperature of the liquid feed stream. In some embodiments, the temperature of the dispersing stream is at least 25° C. lower than the temperature of the liquid feed stream.

In some embodiments, the polymer is miscible in the liquid feed stream and substantially immiscible in the dispersing stream.

In some embodiments, at least about 1 g/L of the polymer is dispersed in the liquid feed stream. In some embodiments, at least about 10 g/L of the polymer is dispersed in the liquid feed stream. In some embodiments, at least about 20 g/L of the polymer is dispersed in the liquid feed stream. In some embodiments, at least about 40 g/L of the polymer is dispersed in the liquid feed stream. In some embodiments, at least about 50 g/L of the polymer is dispersed in the liquid feed stream.

In some embodiments, no more than about 80 g/L of the polymer is dispersed in the liquid feed stream. In some embodiments, no more than about 60 g/L of the polymer is dispersed in the liquid feed stream. In some embodiments, no more than about 50 g/L of the polymer is dispersed in the liquid feed stream.

In some embodiments, the method further comprises applying an energy source to said plurality of nanoparticles dispersed in the dispersing stream for a time that is effective to modify said plurality of nanoparticles.

In some embodiments, the method further comprises recycling a portion of the dispersing stream after intermixing with the liquid feed stream.

Some embodiments include a method of making nanoparticles, comprising: (a) providing a liquid feed stream; and (b) intermixing the liquid feed stream with a dispersing stream, thereby solidifying components of the liquid feed stream into a plurality of nanoparticles dispersed in the dispersing stream, wherein: the dispersing stream has a diameter greater than about 500 μm; the liquid feed stream has a diameter of at least about 100 μm and the nanoparticles have a diameter that is less than about 1000 nm.

Some embodiments include a method of making nanoparticles, comprising: (a) providing a liquid feed stream; and (b) intermixing the liquid feed stream with a dispersing stream, thereby solidifying components of the liquid feed stream into a plurality of nanoparticles dispersed in the dispersing stream, wherein: the dispersing stream has a flow rate of at least about 10 mL/min; and the nanoparticles have a diameter that is less than about 1000 μm.

### BRIEF DESCRIPTION OF THE DRAWINGS

FIGS. 1a-d are illustrations of various examples of Janus particles that may be included in compositions disclosed in the present application.

FIGS. 2a-b are front and side views illustrating an example of a method for making Janus particles.

FIG. 3a-c illustrates an embodiment of a method of forming Janus particles using two liquid feed streams where a vector is in the same direction as the flow direction of the dispersing stream.

FIG. 4a-c illustrates an embodiment of a method of forming Janus particles from two liquid feed streams where a vector is perpendicular to the flow direction of the dispersing stream.

FIG. 5 illustrates the angles θ₁ and θ₂ that may be formed between the liquid feed streams and the dispersing stream.

FIG. 6 illustrates an embodiment that includes a cojoining chamber operably connected to the liquid feed streams and the dispersing stream.

FIG. 7a-c illustrate example configurations having one or more additional feed streams.

FIG. 8a-b illustrate an embodiment of one method for forming nanoparticles.

FIG. 9a illustrates the drug delivery profile for paclitaxel in Janus particles and nanoparticles prepared according to Example 2 and Example 3, respectively.

FIG. 9b illustrates the drug delivery profile for doxorubicin in Janus particles and nanoparticles prepared according to Example 2 and Example 3, respectively.

FIG. 10 includes a graph and SEM images showing nanoparticle diameter for Examples 4-6.

FIG. 11 includes a graph and SEM images showing nanoparticle diameter for Examples 4, 7, and 8.

FIG. 12 includes a graph and SEM images showing nanoparticle diameter for Examples 9-11.

FIG. 13 includes a graph and SEM images showing the nanoparticle size distributions for Example 11 and Comparative Example 1.

### DETAILED DESCRIPTION

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there is a plurality of definitions for a term herein, those in this section prevail unless stated otherwise.

As used herein, a “nanoparticle” refers to any particle having a greatest dimension (e.g., diameter) that is less than about 2500 nm. In some embodiments, the nanoparticle is a solid or a semi-solid. In some embodiments, the nanoparticle is generally centrosymmetric. In some embodiments, the nanoparticle contains a generally uniform dispersion of solid components.

As used herein, a “Janus particle” refers to an inhomogeneous, non-centrosymmetric particle that includes at least two physically or chemically differing components, where at least two of the components are exposed at the surface of the particle. Such exposure is in the form of one or more relatively large continuous surface regions or patches that each occupy a substantial fraction (at least about 5%) of the surface area of the particle. Furthermore, the Janus particle has a total surface area that includes at least about 10% by area of each component that is exposed to the surface. In some embodiments, the Janus particle can be a nanoparticle.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, the terms “pharmaceutical agent,” “drug,” and “active ingredient” refer to any material administered to a subject in a manner intended to produce some biological, beneficial, therapeutic, or other intended effect, such as relief of pain, whether or not approved by a government agency for that purpose.

As used herein, “administration” or “administering” refers to a method of giving a dosage of a pharmaceutically active ingredient to a vertebrate.

As used herein, “therapeutically effective amount” or “pharmaceutically effective amount” is meant an amount of pharmaceutical agent, which has a therapeutic effect. The dosages of a pharmaceutically active ingredient which are useful in treatment are therapeutically effective amounts. Thus, as used herein, a therapeutically effective amount means those amounts of therapeutic agent which produce the desired therapeutic effect as judged by clinical trial results and/or model animal infection studies.

As used herein, a “therapeutic effect” relieves, to some extent, one or more of the symptoms of a disease or disorder. For example, a therapeutic effect may be observed by a reduction in size of a cancerous tumor.

As used herein, the term “imaging agent” is meant to refer to compounds which can be detected by medical imaging techniques. For example, barium sulfate is an X-ray contrast imaging agent.

### Compositions of Janus Particles

Disclosed herein are compositions containing a plurality of Janus particles, each Janus particle having a first component and a second component. The particles may also contain, in some embodiments, two distinct phases.

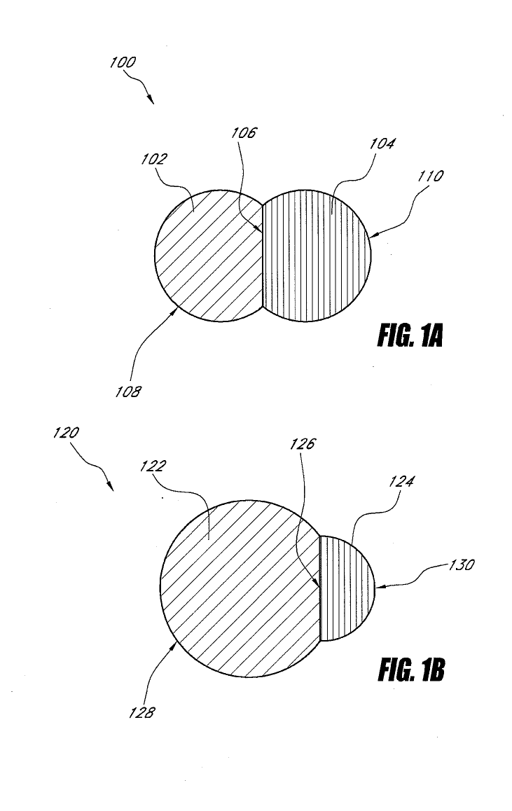

FIGS. 1a-d illustrate various examples of Janus particles that may be present in the compositions described herein. FIG. 1a is a side view of a Janus particle 100 having a first component 102 and a second component 104 that are in contact at an interface 106. The first component 102 and the second component 104 may be about the same size and/or weight. At least a portion 108 of the first component 102 is exposed at the outer surface of the Janus particle 100. Moreover, at least a portion 110 of the second component 104 is also exposed at the outer surface of the Janus particle 100. FIG. 1b shows another example of a Janus particle 120 having a first component 122 and a second component 124. The two components 122,124 similarly contact at an interface 126 and are both exposed at the outer surface 128, 130 of the Janus particle 120; however the two components 122, 124 have a different size and/or weight.

FIG. 1c depicts a three-component Janus particle 140 that may be present in the compositions described herein. The Janus particle 140 includes a first component 142, a second component 144, and a third component 146, where the first component 142 and the second component 144 contact at an interface 148; the second component 144 and third component 146 contact at an interface 150; and the third component 146 and first component 142 contact at an interface 152. At least a portion 154 of the first component 142, a portion 156 of the second component 144 and a portion 158 of the third component 146 are each exposed to the outer surface of the Janus particle 140.

FIG. 1d illustrates a three component Janus particle 160 that may be present in the compositions described herein. The Janus particle 160 has a first component 162, a second component 164, and a third component 166. The first component 162 and second component 164 contact at an interface 168, and the second component 164 and the third component 166 contact at an interface 170; however the third component 166 and first component 162 do not form an interface in this embodiment. As illustrated, the size and/or weight of each component may vary, or alternatively, they may be about the same (not shown). All three components 162, 164, 166 are exposed at the outer surface 172, 174, 176 of the Janus particle 160.

The compositions described herein can include Janus particles having at least two components. For example, the Janus particles may have two, three, four, five or more components. In an embodiment, the Janus particle has two components. Moreover, at least part of the two or more components in the Janus particle can be exposed at the surface of the Janus particle. For example, a Janus particle having three components may have one component that is not exposed at the outer surface and at least part of two components that are exposed at the outer surface of the Janus particle. In some embodiments, all of the components are exposed at the surface of the Janus particle (e.g., the first component and the second component of a two component Janus particle are both exposed).

The Janus particles described herein have a total surface that includes at least portions of the first component and at least portions of the second component. In an embodiment, the total surface area of each Janus particle includes at least 10% by area of the first component that is exposed to the surface of the Janus particle. In another embodiment, the total surface area of each Janus particle includes at least 10% by area of the second component that is exposed to the surface of the Janus particle. In still another embodiment, the total surface area of each Janus particle includes at least 10% by area of, each independently, one or more additional components. The total exposure of each component to the surface of the Janus particle may be further varied. For example, the total surface area of each Janus particle may include at least 15% by area of each component; at least 20% by area of each component; at least 25% by area of each component; at least 30% by area of each component; or at least 40% by area of each component. In some other embodiments, each component exposed to the surface of the Janus particle has an exposed area that is about the same (e.g., a two-component Janus particle may have a total surface area that includes about 50% by area of the first component and about 50% by area of the second component).

Each component in the Janus particle may form a separate, continuous region at the surface of the particle. In an embodiment, each component that is exposed to the surface of the Janus particle independently forms a single, continuous region at the surface of the exposed Janus particle (e.g., components 102 and 104 in Janus particle 100 form separate, continuous regions at the surface of the Janus particle, which meet only at interface 106). In another embodiment, each Janus particle has a total surface area that consists essentially of a total number of continuous regions, where the total number of regions equals the number of components that are exposed to the surface of the Janus particle (e.g., components 102 and 104 form the total surface area in the two-component Janus particle 100 in only two regions, 108 and 110). In other embodiments, each Janus particle has a surface area that consists of a total number of continuous regions, where the total number of regions equals the number of components that are exposed to the surface of the Janus particle.

Embodiments of the Janus particles described herein have a size that is on the scale of about a nanometer or larger. For example, a composition may include Janus particles having an average size of about 10 nm; about 25 nm; about 50 nm, about 100 nm, about 200 nm; about 300 nm; about 500 nm; or about 1000 nm. The Janus particles may have an average size that is less than about 2000 nm; less than about 1000 nm; less than about 500 nm; less than about 300 nm; less than about 200 nm; less than about 100 nm; or less than about 50 nm. The Janus particles may have an average size that is greater than about 10 nm; greater than about 25 nm; greater than about 50 nm; greater than about 100 nm; greater than about 200 nm; greater than about 300 nm; greater than about 500 nm; or greater than about 1000 nm. In an embodiment, the Janus particles have an average size in the range of about 10 nm to about 2000 nm.

The compositions described herein may include Janus particles having a relatively homogeneous size distribution. For example, about 80% of the Janus particles in a composition may have a size within about 30% of the average Janus particle size (e.g., a composition of Janus particles with an average size of 100 nm has 80% of Janus particles in the range of 70 nm to 130 nm). In some embodiments, about 90% of the Janus particles in the composition may have a size within 20% of the average Janus particle size. In other embodiments, about 90% of the Janus particles in the composition may have a size within 10% of the average Janus particle size. In still other embodiments, about 95% of the Janus particles in the composition have a size within 15% of the average Janus particle size.

The Janus particles described herein can have a second component that is substantially different from the first component. For example, the first component can be polyethylene glycol (PEG) and the second component can be polyglycolic acid (PGA). In some embodiments, one or more additional components may be present in the Janus particles that are substantially different than both the first component and the second component. In an embodiment, three or more (e.g., three, four, fives, six, etc.) components present in the Janus particle are substantially different from each other. As an example, Janus particle 140 of FIG. 1c could have the first component 142 be PGA, the second component 144 be PEG, and the third component 146 be polycaprolactone. As would be recognized by those of ordinary skill, the components can be substantially different even if they have the same ingredients. Non-limiting examples of other differences in the components include, but are not limited to: molecular weight, weight percent of ingredients, phase (e.g., crystalline or non-crystalline), microstructure (e.g., grain size), biodegradation properties, and density. In an embodiment, the first component includes at least one ingredient that is not in the second component. In another embodiment, the second component includes at least one ingredient that is not in the first component.

Various ingredients may be incorporated into the two or more components in each Janus particle. For example, one of the components can include one or more polymers that are known to those skilled in the art. The polymer may be a homopolymer, a random copolymer, a block copolymer or a random block copolymer. Moreover, the polymer may be isotactic, syndiotactic or atactic. In some embodiments, the polymer is biodegradable. In some embodiments, the polymer is selected from a polyester, a poly(ortho ester) and a poly(anhydride). In another embodiment, the polymer is a polyester, such as PGA. In still another embodiment, the polymer is a polypeptide, such as polylysine.

Exemplary polymers include, but are not limited to the following: polyethylene glycol (PEG); poly(lactic acid-co-glycolic acid) (PLGA); copolymers of PLGA and PEG; copolymers of poly(lactide-co-glycolide) and PEG; polyglycolic acid (PGA); copolymers of PGA and PEG; poly-L-lactic acid (PLLA); copolymers of PLLA and PEG; poly-D-lactic acid (PDLA); copolymers of PDLA and PEG; poly-D,L-lactic acid (PDLLA); copolymers of PDLLA and PEG; poly(ortho ester); copolymers of poly(ortho ester) and PEG; poly(caprolactone); copolymers of poly(caprolactone) and PEG; polylysine; copolymers of polylysine and PEG; polyethylene imine; copolymers of polyethylene imine and PEG; polyhydroxyacids; polyanhydrides; polyhydroxyalkanoates, poly(L-lactide-co-L-lysine); poly(serine ester); poly(4-hydroxy-L-proline ester); poly[α-(4-aminobutyl)-L-glycolic acid; derivatives thereof; combinations thereof; and copolymers thereof.

In an embodiment, the first component of the Janus particle includes a first polymer. In another embodiment, the first component includes a first polymer having a recurring unit of Formula (I):

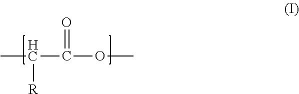

where R can hydrogen or methyl. In some embodiments, the first component includes a first polymer that is PLGA or a PLGA-based copolymer. In some embodiments, the first component includes a first polymer that is not present in the second component.

The molecular weight of the polymer is not particularly limited. In some embodiments, the polymer has an average molecular weight of at least 10,000 Da. In some embodiments, the polymer has an average molecular weight of at least 50,000 Da. In some embodiments, the polymer has an average molecular weight of at least 100,000 Da. In some embodiments, the polymer has an average molecular weight of at least 250,000 Da. In some embodiments, the polymer has an average molecular weight of at least 500,000 Da.

The second component may also include a polymer that is the same or different than the polymer in the first component. In an embodiment, the second component includes a polymer that is absent from the first component. In some embodiments, the second component includes a polymer that is present in the first component.

Various other ingredients may be included in the components depending upon their intended use. The components may include, for example, a pharmaceutical agent or imaging agent. In some embodiments, the first component includes a pharmaceutical agent or an imaging agent. In some embodiments, the second component includes a pharmaceutical agent or an imaging agent. For example, the second component can include an anticancer pharmaceutical agent, such as paclitaxel, or alternatively, a nuclear medicine imaging agent, such as ¹²³I. In an embodiment, the second component includes an ingredient selected from a pharmaceutical agent, an imaging agent and a polymer. The pharmaceutical agent, imaging agent or polymer in the second component may, in some embodiments, be also present in the first component. Alternatively, the first component may, in some embodiments, be substantially free of the pharmaceutical agent, imaging agent or polymer in the second component. In some embodiments, one or more additional components include a second ingredient selected from a pharmaceutical agent, an imaging agent and a polymer.

The Janus particles described herein may have components that are a solid or a gel. In an embodiment, the first component is a solid. In other embodiments, the second component is a solid. In some other embodiments, one or more additional components is a solid. In still other embodiments, all of the components in the Janus particles are solid. Alternatively, one or more components can be a gel.

The relative amount of each component in the Janus particles may be varied depending upon the intended use of the Janus particles. The Janus particles may include the first component in an amount that is at least about 30% by weight; at least about 70% by weight; or at least about 90% by weight. Furthermore, the Janus particle may include the first component in an amount that is no more than about 99.5%; no more than about 95% by weight; or no more than about 80% by weight. In an embodiment, the Janus particles include a first component in an amount in the range of about 30% to about 90% by weight. Similarly, various amounts of the second component can be included in the Janus particles. The Janus particles may include the second component in an amount of at least about 0.5% by weight; at least about 5% by weight; at least about 10% by weight; or at least about 50% by weight. Also, the second component may be included in the Janus particles in an amount that is no more than about 20% by weight; no more than about 15% by weight; or no more than about 5% by weight. In another embodiment, the Janus particles include a second component in an amount in the range of about 10% to about 70% by weight.

The concentration of Janus particles in the compositions described herein are not particularly limited, and can be modified by concentrating or diluting compositions as desired. The composition may include Janus particles at a concentration of at least about 0.1 parts per million by weight (ppm). Alternatively, the composition may include at least about 1 ppm of Janus particles; at least about 10 ppm of Janus particles; or at least about 100 ppm of Janus particles. Also, the compositions may include at least about 1 mg of Janus particles; at least about 10 mg of Janus particles; at least about 100 mg of Janus particles; at least about 1 g of Janus particles; or at least about 100 g of Janus particles.

### Methods of Making Janus Particles

Also disclosed herein are methods of making Janus particles, including methods of making the multi-component Janus particles described above. The method may include providing at least a first liquid feed stream and a second liquid feed stream; and intermixing the first liquid feed stream and the second liquid feed stream with a dispersing stream, thereby solidifying components of the first liquid feed stream and the second liquid feed stream into a plurality of Janus particles dispersed in the dispersing stream. In an embodiment, the first liquid feed stream includes a first component and the second liquid feed stream includes a second component that is substantially different from the first component. In other embodiments, the first liquid feed stream includes a first component that is a first polymer. In another embodiment, the plurality of Janus particles each include the first component and the second component.

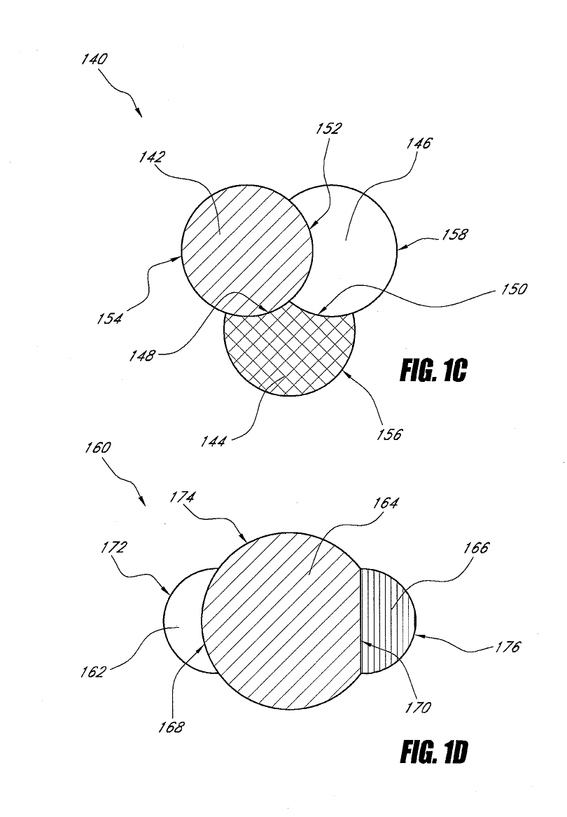

FIGS. 2a-b illustrate an embodiment of a method of making a Janus particle. FIG. 2a is a front view of a first liquid feed stream 200 and a second liquid feed stream 205 that flow through a first channel 210 and a second channel 215, respectively. Both feed streams 200, 205 are output from the channels 210, 215 so that the first liquid feed stream 200 and the second liquid feed stream 205 contact each other as illustrated. Moreover, the first liquid feed stream 200 and the second liquid feed stream 205 exit their respective channels and contact a dispersing stream 220, which flows within a dispersing channel 222 in a direction out of the page. FIG. 2b is a side view of the configuration of FIG. 2a, where the dispersing stream 220 flows from left to right. The first liquid feed stream 200 and the second liquid feed stream 205 solidify upon contacting the dispersing stream 220 to form a discrete Janus particle 225 having a first component 230 (from the first liquid feed stream 200) and a second component 235 (from the second liquid feed stream 205) that form separate portions of the Janus particle 225. The two liquid feed streams 200, 205 and the dispersing stream 220 may, in some embodiments, continuously flow, such that a plurality of Janus particles form in the dispersing stream.

Those skilled in the art will understand that the two liquid feed streams can be configured so a first component of the first liquid feed stream and a second component of the second liquid feed stream solidify before substantial intermixing with one another. In an embodiment, the two liquid feed streams solidify into Janus particles that include a first component from the first liquid feed stream and a second component from the second liquid feed stream. In another embodiment, the Janus particles include the first component and the second component in separate portions of the Janus particle. In other embodiments, at least part of the first component is exposed at the surface of the Janus particle. In some other embodiments, at least part of the second component is exposed at the surface of the Janus particle. In still another embodiment, the Janus particles include an interface between the first component and the second component. For example, the liquid feed streams may solidify into a plurality of Janus particles, each having a structure as illustrated in FIG. 1a-d. Thus, the methods can be used to make the Janus particles and compositions described herein.

The two liquid feed streams can be substantially different from each other. In some embodiments, the first liquid feed stream includes a first component that is substantially different from a second component included in the second liquid feed stream. In an embodiment, the first liquid feed stream has a first component that includes a first polymer that is substantially different from a second component in the second liquid feed stream, wherein the second component includes an ingredient selected from a pharmaceutical agent, an imaging agent, and a polymer.

Various ingredients may be used in each component of the liquid feed streams, including any of those described in the composition of Janus particles disclosed above. The liquid feed stream can also include one or more solvents, such as an organic solvent. The solvent(s) in each liquid feed stream can be the same, or they can be different. Some examples of solvents that may be suitable for use in the liquid feed stream include, but are not limited to: 1,4 dioxane, tetrahydrofuran (THF), acetone, acetonitrile, dimethyl sulfoxide (DMSO), dimethylformamide (DMF), acids, alcohols (e.g., C₁-C₈ alcohols, such as methanol, ethanol, isopropanol, and octanol), and combinations thereof. In an embodiment, the first liquid feed stream can include a solvent that is at least partially miscible in the dispersing stream. In another embodiment, the second liquid feed stream can include a solvent that is at least partially miscible in the dispersing stream. In some embodiments, the first liquid feed stream can include a solvent that is miscible in the dispersing stream. In some other embodiments, the second liquid feed stream can include a solvent that is miscible in the dispersing stream.

The concentration of components within the first and second liquid feed streams will vary depending upon factors such as the solubility, molecular weight, relative amount of components intended for each Janus particle, and other factors that will be recognized by those skilled in the art guided by the teachings provided herein. In an embodiment, at least 1 g/L of a first component is dispersed in the first liquid feed stream. The first component can, for example, be a polymer (e.g., PGA, PLA, PGLA, PEG, etc.). In some embodiments, at least 1 g/L of a second component is dispersed in the second liquid feed stream. The second component may be selected from a polymer, a pharmaceutical agent and an imaging agent. For example, the second component may be a polymer that is different from any polymers in the first liquid feed stream.

Additionally, the liquid feed streams may be configured to adjust their viscosities. In an embodiment, the viscosity of the first liquid feed stream is selected so that the first liquid feed stream flows at a rate of at least 1 μL/hr. when the stream is under a pressure of no more than 7 MPa. In other embodiments, the viscosity of the second liquid feed stream is selected so that the second liquid feed stream flows at a rate of at least 1 μL/hr. when the stream is under a pressure of no more than 7 MPa. In another embodiment, the viscosity of the dispersing stream is selected so that the dispersing stream flows at a rate of at least 2 μL/hr. when the stream is under a pressure of no more than 7 MPa.

One or more liquid feed streams may include components that are at least partially dissolved in a solvent. Alternatively, one or more liquid feed streams may include an emulsion of at least one component in a carrier. In an embodiment, the first liquid feed stream includes a first component that is dissolved in a solvent. In some embodiments, the second liquid feed stream includes a second component that is dissolved in a solvent. Moreover, the first component may be miscible in the first liquid feed stream and substantially immiscible in the dispersing stream. Also, the second component may be miscible in the second liquid feed stream and substantially immiscible in the dispersing stream.

Moreover, the contents of the dispersing stream are not particularly limited, and may be modified to alter the properties of the Janus particle. In an embodiment, the first component of the first liquid feed stream and the second component of the second liquid feed stream are both substantially immiscible in the dispersing stream. In another embodiment, the first component and the second component are both immiscible in the dispersing stream. Various solvents may be included in the dispersing stream, such as an organic solvent or water. In some embodiments, the solvent in the dispersing stream is different than any solvents in the first and second liquid feed streams. In a preferred embodiment, the solvent is water.

Without being bound to any particular theory, it is believed that solidification of the components of the first liquid feed stream and the second liquid feed stream is caused, at least in part, by diffusion of the solvents in the first liquid feed stream and the second liquid feed stream into the dispersing stream. Thus, selection of the solvents for the liquid feed streams and the dispersing stream can influence the resultant Janus particle properties. As would be appreciated by a person of ordinary skill in view of the guidance provided herein, a chemical reaction may also be desirable in some instances to solidify the components. In an embodiment, the solidifying of the components of the first liquid feed stream and the second liquid feed stream comprises diffusion of the first solvent and the second solvent into the dispersing stream. In another embodiment, the solidifying of the components of the first liquid feed stream and the second liquid feed stream consists essentially of diffusion of the first solvent and the second solvent into the dispersing stream. Furthermore, in some embodiments, the solidification of the components does not include a cross-linking reaction or a polymerization reaction. In still another embodiment, the solidification of the components does not include a chemical reaction, such as a polymerization reaction, a cross-linking reaction, etc.

Other additives can be included to improve the properties of the dispersing stream and/or modify the Janus particles. Exemplary additives include, but are not limited to, polymers, salts, surfactants, plasticizers, antimicrobial agents, thickening agents and the like. In an embodiment, the dispersing stream includes a polymer, such as polyvinyl alcohol. For example, the dispersing stream can be water having 1% polyvinyl alcohol by weight.

The temperature of the dispersing stream can be different than the first or second liquid feed stream. The temperature of the dispersing stream may, for example, be at least 1° C. lower than at least one of the first liquid feed stream or the second liquid feed stream. In some embodiments, the temperature of the dispersing stream is at least 5° C. lower than at least one of the first liquid feed stream or the second liquid feed stream. In other embodiments, the temperature of the dispersing stream is at least 10° C. lower than at least one of the first liquid feed stream or the second liquid feed stream. In another embodiment, the temperature of the dispersing stream is at least 25° C. lower than at least one of the first liquid feed stream or the second liquid feed stream.

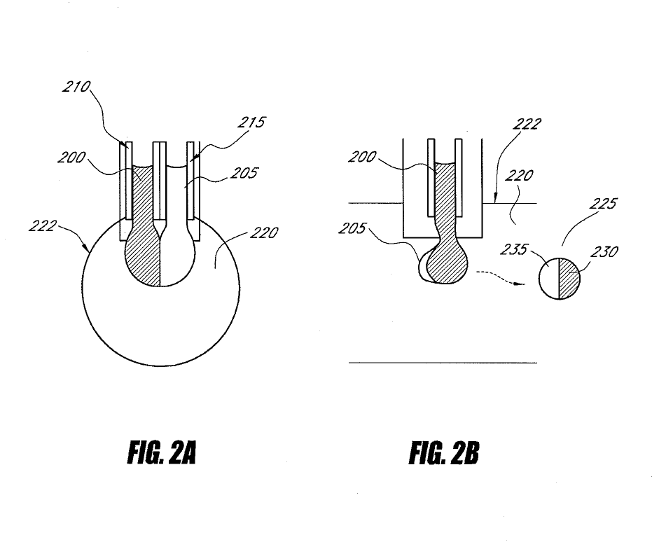

The methods described herein may be practiced in numerous configurations of the liquid feed streams to obtain the desired Janus particles. For example, FIG. 3a-c illustrates an embodiment of a suitable configuration for the liquid freed streams. FIG. 3a is a side view of the configuration, where a first liquid feed stream 305 flows out of a first outlet 310 and a second liquid feed stream 315 also flows out of a second outlet 320. The first and second outlets 310, 320 are positioned near each other so that the first and second liquid feed streams 305, 315 contact each other upon exiting the outlet. Both liquid feed streams contact the dispersing stream 325, which flows from left to right. FIG. 3b is a front view of the same configuration, where the dispersing stream 325 flows out of the page. FIG. 3c is a view along the axis of the liquid feed streams 305, 315, such that the liquid feed streams 305, 315 flow out of the page and the dispersing stream 325 flows from left to right. The first outlet 310 and the second outlet 320 each have a first center 330 and a second center 340, respectively, such that a vector 345 exists between the two centers. In this configuration, the vector and the dispersing stream flow direction are substantially parallel and in the same direction (i.e., form an angle of about 0 degrees).

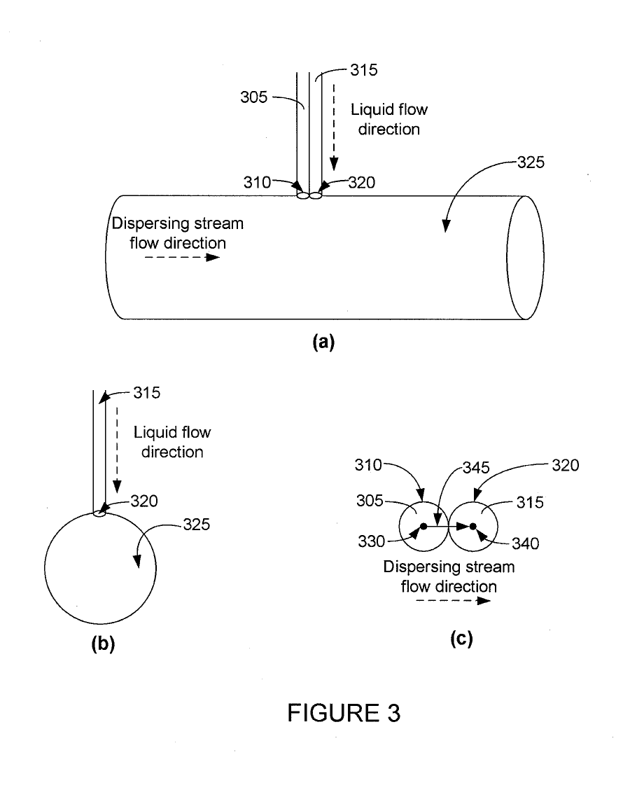

An alternative configuration is illustrated in FIGS. 4a-c, which has numbered items 405 through 445 that correspond to items 305 through 345 in FIGS. 3a-c, respectively (e.g., 325 and 425 are both dispersing streams). The orientations of the two liquid feed streams 405, 415 with respect to the dispersing channel are such that the vector 445 is perpendicular to the dispersing stream flow direction (i.e., form an angle of about 90 degrees).

Thus, the vector and the dispersing stream flow direction form an angle ψ. In some embodiments, ψ is in the range of about −175 degrees to about 175 degrees. In other embodiments, ψ is in the range of about 5 degrees to about 355 degrees. In still another embodiment, ψ is in the range of about 45 degrees to about 135 degrees. In an embodiment, ψ is in the range of about 225 degrees to about 315 degrees. The angle ψ may also be about 0 degrees; about 90 degrees; about 180 degrees; or about 270 degrees.

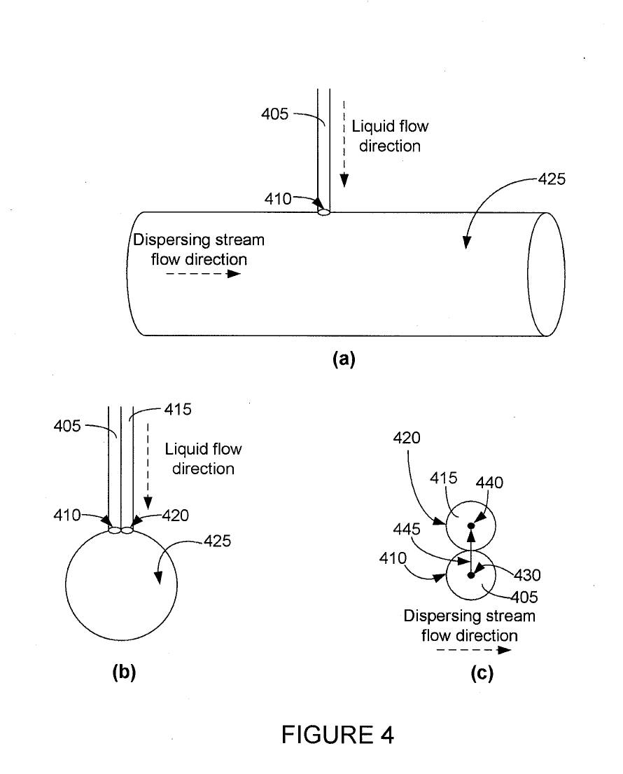

Additionally, as shown in FIG. 5, each liquid feed stream 505, 515 may be independently oriented to form an angle θ with the dispersing stream 525. The first liquid feed stream 505 may form an angle θ₁ with the dispersing stream 525, and the second liquid feed stream 515 may form an angle θ₂ with the dispersing stream 525 to from an angle θ₂. In an embodiment, the angle θ₁ is in the range of about 5 degrees and about 175 degrees. In another embodiment, the angle θ₁ is in the range of about 0 degrees and about 170 degrees. In some embodiments, the angle θ₁ is in the range of about 10 degrees and about 180 degrees. In some more embodiments, the angle θ₁ is in the range of about 45 degrees and about 135 degrees. The angle θ₁ may be about 0 degrees, about 90 degrees; or about 180 degrees. In still other embodiments, the angle θ₂ is in the range of about 5 degrees and about 175 degrees. In an embodiment, the angle θ₂ is in the range of about 0 degrees and about 170 degrees. In other embodiments, the angle θ₂ is in the range of about 10 degrees and about 180 degrees. In some embodiments, the angle θ₂ is in the range of about 45 degrees and about 135 degrees. The angle θ₂ may also be about 0 degrees; about 90 degrees; or about 180 degrees.

As would be appreciated by a person of ordinary skill in view of the guidance provided herein, the angles θ₁, θ₂, and ψ can be modified to optimize various properties of the Janus particles, such as, for example, the shape and/or size of the Janus particles.

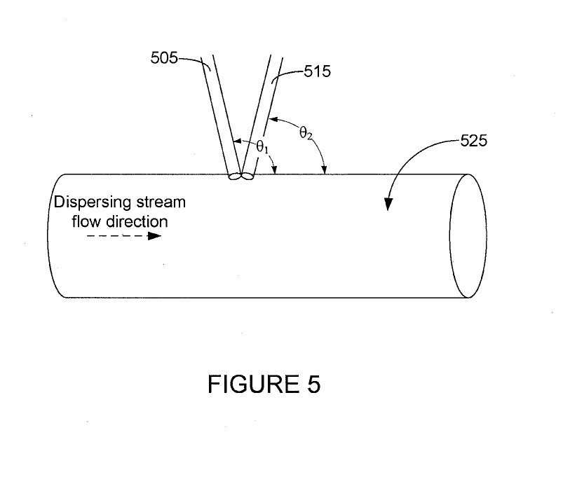

The order in which the first liquid feed stream, the second liquid feed stream, and the dispersing stream contact each other can also be configured to advantage. This may be achieved, for example, by modifying the structural arrangement of any channels that the liquids flow through. In an embodiment, a portion of the first liquid feed stream contacts a portion of the second liquid feed stream before the portion of the first liquid feed stream and/or the portion of the second liquid feed stream contacts the dispersing stream. As an example shown in FIG. 6, the first liquid feed stream may flow in a first channel that is adjacent to a second channel in which the second liquid feed stream flows. The channels may merge into a common channel, or a cojoining chamber, such that the two liquid feed streams contact each other before contacting the dispersing stream in the dispersing channel. The cojoining chamber may have a length in the range of about 10 nm to about 100 μm or in the range of about 10 μm to about 100 μm. In another embodiment, a portion of the first liquid feed stream, a portion of the second liquid feed stream and the dispersing stream all initially contact each other at about the same time. For example, the liquid feed streams may be configured without a conjoining chamber, such as shown in FIG. 3a. In still another embodiment, a portion of the first liquid feed stream and/or a portion of the second liquid feed stream contact the dispersing stream before the portion of the first liquid feed stream contacts the portion of the second liquid feed stream.

The properties of the Janus particles may further be controlled by the size and flow rate of the liquid feed streams. The first liquid feed stream may have a first diameter that is in the range of about 1 μm to about 1 mm; whereas the second liquid feed stream may also have a second diameter that is in the range of about 1 μm to about 1 mm. The liquid feed streams may both independently have a diameter that is at least about 10 μm; at least about 50 μm; at least about 100 μm; at least about 250 μm; at least about 500 μm; or at least about 750 μm. Also, the liquid feed streams may both independently have a diameter that is no more than about 1 mm; no more than about 750 μm; no more than about 500 μm; no more than about 250 μm; no more than about 100 μm; or no more than about 50 μm. Furthermore, the dispersing stream can have a third diameter that is at least about 2 times larger than the first diameter and the second diameter. For example, the first and second diameters may both be about 500 μm and the third diameter is about 2 mm. Alternatively, the third diameter can be at least about 5 times larger than the first diameter and the second diameter.

The first liquid feed stream and the second liquid feed stream may both independently have a flow rate in the range of about 1 μL/hr. to about 100 mL/min. The first and second liquid feed stream may both independently have a flow rate that is at least about 1 μL/hr; at least about 10 μL/hr; at least about 1 μL/min; at least about 10 μL/min; or at least about 100 μL/min. Furthermore, the first and second liquid feed stream may both independently have a flow rate that is no more than about 100 mL/min; no more than about 100 μL/min; no more than about 10 μL/min; or no more than about 10 μL/hr. In some embodiments, the flow rate of the first and second liquid feed streams is about the same. In another embodiment, the first and second liquid feed streams have different flow rates. Also, the dispersing stream may have a flow rate that is in the range of about 2 times greater and about 10 times greater than the first liquid feed stream. In an embodiment, the dispersing stream may also have a flow rate that is in the range of about 3 times greater and about 6 times greater than the first liquid feed stream.

The plurality of Janus particles dispersed in the dispersing stream may optionally be subjected to various post-formation steps and/or treatments. For example, the plurality of Janus particles dispersed in the dispersing stream may be subjected to an energy source, such as ultraviolet radiation, for a time that is effective to alter the chemical properties of the Janus particles (e.g., cross-linking or polymerizing components). The post-formation steps and/or treatments may be applied in a continuous manner to the Janus particles dispersed in the dispersing stream. As an example, ultra-violet radiation may be applied to a region where the dispersing stream, which includes dispersed Janus particles, flows thereby irradiating all or most of the Janus particles formed.

In an embodiment, the Janus particles are subjected to an isolating step, whereby the Janus particles are isolated from the dispersing stream. Various method of isolating Janus particles are known by those of ordinary skill, such as filtration, sedimentation, centrifugation, decantation, drying, magnetic separation, and the like. In an embodiment, the isolation step is completed by filtering the dispersing stream. For example, the dispersing stream may flow through a filter that isolates the Janus particles formed in the dispersing stream. The filtration may be completed in a continuous manner by having the filter operably connected to the dispersing stream containing the Janus particles.

The method may also include recycling a portion of the dispersing stream after intermixing the dispersing stream with the first and second liquid feed streams. For example, after Janus particles are formed in the dispersing stream, the dispersing stream can optionally be subjected to an isolation step, and then portions of the dispersing stream reflow past the liquid feed streams at least a second time. In an embodiment, substantially all of the dispersing stream is recycled after intermixing with the first and second liquid feed streams. The recycling may be completed so that the dispersing stream flows in a closed loop.

Some embodiments disclosed herein include one or more additional liquid feed streams. In an embodiment, the one or more liquid feed streams may be configured to intermix with the dispersing stream so that additional components solidify into Janus particles that also include the first and second components from the first and second liquid feed streams. For example, a third liquid feed stream can have an outlet adjacent to the first and second liquid feed streams. The third liquid feed stream contacts the first and second liquid feed streams and the dispersing stream to form a Janus particle having three components (e.g., as shown in FIG. 1c as Janus particle 140).

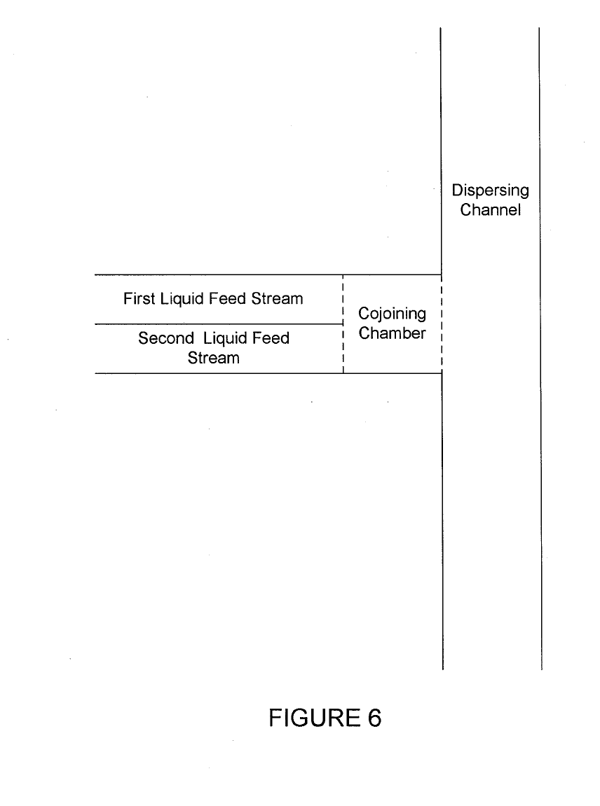

The one or more additional liquid feed streams may also be configured so the additional components intermix with the dispersing stream to form Janus particles other than those formed by the first and second liquid feed streams. Thus, the one or more additional liquid feed streams can form additional Janus particles in the dispersing stream at about the same time that the first liquid feed stream and the second liquid feed stream form Janus particles in the dispersing stream. In an embodiment, the Janus particles formed by the one or more additional liquid feed streams are substantially the same as those formed by the first and second liquid feed streams. FIGS. 7a-c show exemplary configurations of the one or more additional feed streams that form separate Janus particles. FIG. 7a is a side view of a series configuration, where a total of four liquid feed streams can be used to form two Janus particles at about the same time. FIG. 7b-c are both different views of an axial configuration having eight liquid feed streams that can form four Janus particles at about the same time. FIG. 7b is a side view showing four pairs of liquid feed streams positioned at about the same distance along the flow path of the dispersing stream. FIG. 7c is a view along the axis of the dispersing stream that shows the pairs of liquid feed streams located at different radial positions about the axis of the dispersing stream flow direction. In an embodiment, the axial configuration has the pairs of liquid feed streams located symmetrically about the axis of the dispersing stream. As would be recognized by those of ordinary skill in view of the guidance provided herein, the liquid feed streams may be configured to be both in series and have an axial arrangement. For example, there may be 8 liquid streams positioned along the dispersing stream in an axial configuration, which is followed by 8 more liquid feed streams positioned further along the dispersing stream in an axial configuration.

### System for Making Janus Particles

Also disclosed herein are systems for making Janus particles. In particular, the system may be used to make the Janus particles disclosed herein and/or carry out the methods disclosed herein. The system can include a first feed channel (e.g., channel 210 in FIG. 2), a second feed channel (e.g., channel 215 in FIG. 2) and a dispersing channel (e.g., channel 222 in FIG. 2). In an embodiment, the first feed channel, the second feed channel, and the dispersing channel can be configured to have the first liquid feed stream, the second liquid feed stream, and the dispersing stream, as described above with respect to the method of making Janus particles, flow through the respective channels.

The first feed channel can have a first outlet that is operably connected to the dispersing channel. Also, the second feed channel can have a second outlet that is operably connected to the dispersing channel. In an embodiment, the first outlet and the second outlet can be about 5 mm apart or less. In some embodiments, the first outlet and the second outlet can be about 1 mm apart or less. Meanwhile, the first outlet can be within about 1 mm of the dispersing channel. Also, the second outlet can be within about 1 mm of the dispersing channel.

As disclosed above with respect to the method of making Janus particles, a co-joining channel may be included within the system. The first outlet and the second outlet can be operably connected to a cojoining channel that is operably connected to the dispersing channel (see, e.g., FIG. 6). Thus, the cojoining channel is configured so that any contents flowing in the first feed channel and the second feed channel contact before contacting the contents of the dispersing channel. In an embodiment, the cojoining channel is configured so that the first outlet or the second outlet is at least about 10 nm from the dispersing channel. In another embodiment, the cojoining channel is configured so that the first outlet or the second outlet are in the range of about 10 nm to about 100 μm from the dispersing channel. In still another embodiment, the cojoining channel is configured so that the first outlet or the second outlet are in the range of about 1 μm to about 100 μm from the dispersing channel.

The system may also include one or more additional feed channels that are configured to have one or more additional liquid feed streams, as described above, flow through the channel. In an embodiment, the one or more additional feed channels have one or more additional outlets operably connected to the dispersing channel. In some embodiments, the one or more additional liquid feed channels include a third feed channel having a third outlet, and a fourth feed channel having a fourth outlet, where the third outlet and the fourth outlet are within about 1 mm. In some embodiments, the one or more additional feed channels include a third channel having a third outlet that is within about 1 mm of the first outlet or the second outlet.

The feed channels may be prepared using various methods known by those skilled in the art. Non-limiting examples of forming the channels include lithography, embossing, or molding. Also, the materials for making the channels is not particularly limited, however the channel may include a polymer, such as polyvinylchloride (PVC), or steel, such as stainless steel.

The first channel may have a first diameter that can be the same as those described above with respect to the first liquid feed streams. For example, the first channel may have a diameter in the range of about 10 μm to about 1 mm. Similarly, the second channel may have a diameter that can be the same as those described above with respect to the second liquid feed stream. Finally, the dispersing stream has a third diameter that can be at least about 2 times larger than the first diameter.

Various other devices may be operably connected to the system. The system can include one or more pumps configured to displace a substance in the first feed channel, the second feed channel, and/or the dispersing channel. Also, the system may include an isolating means, such as a filter, or any other device disclosed herein, that is operably connected to the dispersing channel. Furthermore, the system may include one or measuring devices, operably connected to the first feed channel, the second feed channel, the dispersing channel, one or more pumps, and/or an isolating means. For example, a temperature coupling may be configured to measure the temperature of the dispersing stream, or a flow meter may be configured to measure the flow rate of the first liquid feed stream in the first channel. The system may also include a processor that is in communication with the one or more pumps and/or one or more measuring devices.

Method of Treatment using Janus Particles

The application also includes methods of treating a mammal with a disease by administering pharmaceutically effective amounts of a composition of Janus particles. The composition of Janus particles may be the same as those described herein and may be used for drug delivery of a pharmaceutical agent to a mammal. In an embodiment, the composition of Janus particles has a first component that includes a pharmaceutical agent. In another embodiment, the composition of Janus particles has a second component that is substantially different from the first component.

In some embodiments, the second component includes a second pharmaceutical agent. The second pharmaceutical agent may be the same as, or different than, the pharmaceutical agent in the first component. For example, a Janus particle may include two components that have the same pharmaceutical agent. However, the components may be substantially different because the relative amount of pharmaceutical agent is different (e.g., 10% by weight pharmaceutical agent in a first component and 50% by weight pharmaceutical agent in a second component). Or the components may be different because each component includes a different polymer (e.g., a first component includes PLGA and a second component includes PGA). Alternatively, in some embodiments, the first and second components include different pharmaceutical agents (e.g., paclitaxel in a first component and doxorubicin in a second component).

The composition of Janus particles may be the same as those described herein within respect to the composition of Janus particles. For example, the Janus particles may have an average size in the range of about 10 nm to about 2000 nm. In an embodiment, the Janus particles may have at least part of the first component exposed to the surface of the Janus particle. In another embodiment, the Janus particles may have at least part of the second component exposed to the surface of the Janus particle.

The type of disease that may be treated using the composition of Janus particles is generally not limited, so long as an appropriate pharmaceutical agent is included for delivery within the Janus particles. In an embodiment, the pharmaceutical agent may be an anti-thrombotic agent (e.g., heparin, hirudin analogs like hirulog, inhibitors of factor Xa, inhibitors of thrombin, etc), an anti-platelet agent (e.g., GPIIb-IIIa antagonists, prostaglandins and prostaglandin analogs), a thrombolytic agent (e.g., plasminogen activator), an anti-proliferative agent, a chemotherapeutic agent, an anti-biotic agent, agents that induce cholesterol efflux from macrophages (e.g., agonist of LXR), or an inhibitor of fatty acid biosynthesis (e.g., inhibitors of fatty acid synthase, acetyl coA carboxylase, ATP citrate lyase).

In some embodiments, the Janus particles are used to treat cancer or a proliferative disease. For example, the pharmaceutical agent can be an anticancer drug, such as paclitaxel. Other examples of anticancer drugs include, but are not limited to, cisplatin, oxaliplatin, carboplatin, doxorubicin, a camptothecin, methotrexate, vinblastine, etoposide, docetaxel hydroxyurea, celecoxib, fluorouracil, busulfan, imatinib mesylate, alembuzumab, aldesleukin, and cyclophosphamide. In some embodiments, the Janus particles include a second pharmaceutical agent that is also an anticancer drug. Thus, for example, Janus particles may include a first component having paclitaxel and a second component having doxorubicin.

Meanwhile, various antiproliferative agents may be used, such as angiotensin converting enzyme (ACE) inhibitors (e.g., angiopeptin, captopril, cilazapril, and lisinopril), calcium channel blockers (e.g., nifedipine), colchicine, fibroblast growth factor (FGF) antagonists, omega 3-fatty acid, histamine antagonist, lovastatin, monoclonal antibodies (e.g., PDGF receptors), nitroprusside, phosphodiesterase inhibitors, prostaglandin inhibitor, seramin, serotonin blockers, steroids, thioprotease inhibitors, triazolopyrimidine, and nitric oxide.

Some embodiments of the present application are advantageous because they permit forming (and administering) Janus particles containing two pharmaceutical agents with disparate solubility profiles. As an example, the Janus particle may contain a first pharmaceutical agent that is hydrophobic (e.g., paclitaxel) and a second pharmaceutical agent that is hydrophilic (e.g., doxorubicin). These Janus particles may be desirable because they can provide targeted delivery of paclitaxel and doxorubicin to generally the same region (e.g., a particular tissue) despite their disparate solubility properties.

In some embodiments, the Janus particles include two pharmaceutical agents having different partition coefficients. Typically, the partition coefficient (Log P) corresponds to the logarithmic value of the ratio at which a compound partitions between octanol and water solutions. Partition coefficients can be readily determined using routine experimental procedures or by referencing various publications. See e.g., O'Neil, M., The Merck Index: An Encyclopedia of Chemicals, Drugs, and Biologicals, Merck, 14^{th} ed. (2006). In some embodiments, the difference between the partition coefficient of the first pharmaceutical agent and the partition coefficient of the second pharmaceutical agent is at least about 0.5. In some embodiments, the difference between the partition coefficient of the first pharmaceutical agent and the partition coefficient of the second pharmaceutical agent is at least about 1. In some embodiments, the difference between the partition coefficient of the first pharmaceutical and the partition coefficient of the second pharmaceutical agent is at least about 1.5. As one example, paclitaxel and doxorubicin have partition coefficients of about 3.6 and about 0.4, respectively.

Moreover, in some embodiments, the first pharmaceutical agent has a partition coefficient that is less than about 2.5. In some embodiments, the first pharmaceutical agent has a partition coefficient that is less than about 2. In some embodiments, the first pharmaceutical agent has a partition coefficient that is less than about 1.5. In some embodiments, the first pharmaceutical agent has a partition coefficient that is less than about 1. In some embodiments, the second pharmaceutical agent has a partition coefficient that is greater than about 2.5. In some embodiments, the second pharmaceutical agent has a partition coefficient that is greater than about 3. In some embodiments, the second pharmaceutical agent has a partition coefficient that is greater than about 3.5.

Table 1 includes additional non-limiting examples of pharmaceutical agents that may be incorporated into Janus particles and provides the partition coefficient for each pharmaceutical agent.

TABLE 1
Non-limiting Examples of Pharmaceutical Agents
and their Respective Partition Coefficients.

| Pharmaceutical | Partition |
|---|---|
| Agent | Coefficient |
| Fulvestrant (SERD) | 8.5 |
| Rapamycin | 6.9 |
| Everolimus | 6.8 |
| Temsirolimus | 6.8 |
| Raloxifene | 6.3 |
| Toremifene | 5.7 |
| Lapatinib | 5.1 |
| Irofulven | 4.7 |
| Gefitinib | 4.5 |
| Erlotinib HCl | 3.9 |
| Dasatinib | 3.9 |
| Exemestane | 3.8 |
| Paclitaxel | 3.6 |
| Anastrozole | 3.5 |
| 17-AAG | 3.4 |
| Dovitinib | 3.3 |
| Formestane | 3.1 |
| Entinostat | 3.1 |
| Letrozole | 3.1 |
| Lonafarnib | 3.1 |
| Tamoxifen | 2.9 |
| Panobinostat | 2.8 |
| Mocetinostat | 2.7 |
| Metoprine | 2.6 |
| Valproic acid | 2.6 |
| Tiludronate | 2.3 |
| Vorozole | 2.2 |
| Dacinostat | 2.1 |
| Vorinostat (SAHA) | 1.4 |
| Veliparib (ABT-888) | 1 |
| Belinostat | 1 |
| doxorubicin | 0.4 |
| cyclosphosphamide | 0.3 |
| etoposide | 0.06 |
| Bortezomib | −0.4 |
| 5-FU | −0.6 |
| methotrexate | −0.7 |
| Indinavir | −1.2 |
| fludarabine | −1.3 |
| gemcitabine | −1.4 |
| cisplatin | −1.6 |
| cisplatin | −1.6 |
| Ibandronate sodium | −4.4 |
| Clodronate | −5.6 |
| Risedronate | −5.8 |
| Etidronate | −6 |
| Alendronate | −6.5 |
| Pamidronate | −7 |

The concentration of the optional first pharmaceutical agent in the Janus particles is not particularly limited. In some embodiments, the Janus particles include less than about 25% by weight of the first phaimaceutical agent. In some embodiments, the Janus particles include less than about 10% by weight of the first pharmaceutical agent. In some embodiments, the Janus particles include less than about 5% by weight of the first pharmaceutical agent. In some embodiments, the Janus particles include less than about 3% by weight of the first pharmaceutical agent. In some embodiments, the Janus particles include at least about 0.1% by weight of the first pharmaceutical agent. In some embodiments, the Janus particles include at least about 0.5% by weight of the first pharmaceutical agent. In some embodiments, the Janus particles include at least about 1% by weight of the first pharmaceutical agent. In some embodiments, the Janus particles include at least about 3% by weight of the first pharmaceutical agent.

Similarly, the concentration of the optional second pharmaceutical agent in the Janus particles is not particularly limited. In some embodiments, the Janus particles include less than about 25% by weight of the second pharmaceutical agent. In some embodiments, the Janus particles include less than about 10% by weight of the second pharmaceutical agent. In some embodiments, the Janus particles include less than about 5% by weight of the second pharmaceutical agent. In some embodiments, the Janus particles include less than about 3% by weight of the second pharmaceutical agent. In some embodiments, the Janus particles include at least about 0.1% by weight of the second pharmaceutical agent. In some embodiments, the Janus particles include at least about 0.5% by weight of the second pharmaceutical agent. In some embodiments, the Janus particles include at least about 1% by weight of the second pharmaceutical agent. In some embodiments, the Janus particles include at least about 3% by weight of the second pharmaceutical agent.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. (See e.g., Fingl et al. 1975, in “The Pharmacological Basis of Therapeutics”, which is hereby incorporated herein by reference in its entirety, with particular reference to Ch. 1, p. 1). The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

Although the exact dosage will be determined on a drug-by-drug basis, in most cases, some generalizations regarding the dosage can be made. The daily dosage regimen for an adult human patient may be, for example, an oral dose of between 0.01 mg and 3000 mg of each active ingredient, preferably between 1 mg and 700 mg, e.g. 5 to 200 mg. The dosage may be a single one or a series of two or more given in the course of one or more days, as is needed by the patient. In some embodiments, the compounds will be administered for a period of continuous therapy, for example for a week or more, or for months or years.

In instances where human dosages for compounds have been established for at least some condition, those same dosages my be used, or dosages that are between about 0.1% and 500%, more preferably between about 25% and 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Alternatively dosages may be based and calculated upon the surface area of the patient, as understood by those of skill in the art.

### Methods of Making Nanoparticles

Also disclosed herein are methods of making nanoparticles. In some embodiments, the method includes providing a liquid feed stream; intermixing the liquid feed stream with a dispersing stream, thereby solidifying components of the liquid feed stream into a plurality of nanoparticles dispersed in the dispersing stream.

The nanoparticles may be formed using generally the same systems and methods as those disclosed above with respect to Janus particles. Nanoparticles may be formed using the above-described systems and methods, for example, by configuring the liquid feed streams so that components from each liquid feed stream solidify into separate particles, rather than combining into a Janus particle. As an example, each liquid feed stream can be appropriately spaced apart so that components from each liquid feed stream form separate nanoparticles. One example of a system for forming nanoparticles might include two or more liquid feed streams that intersect the dispersing stream, where each feed stream is at least about 1 mm apart. In some embodiments, each liquid feed stream is at least about 5 mm apart. In some embodiments, each liquid feed stream is at least about 10 mm apart. Alternatively, a system could include only a single liquid feed stream to form nanoparticles that are not Janus particles.

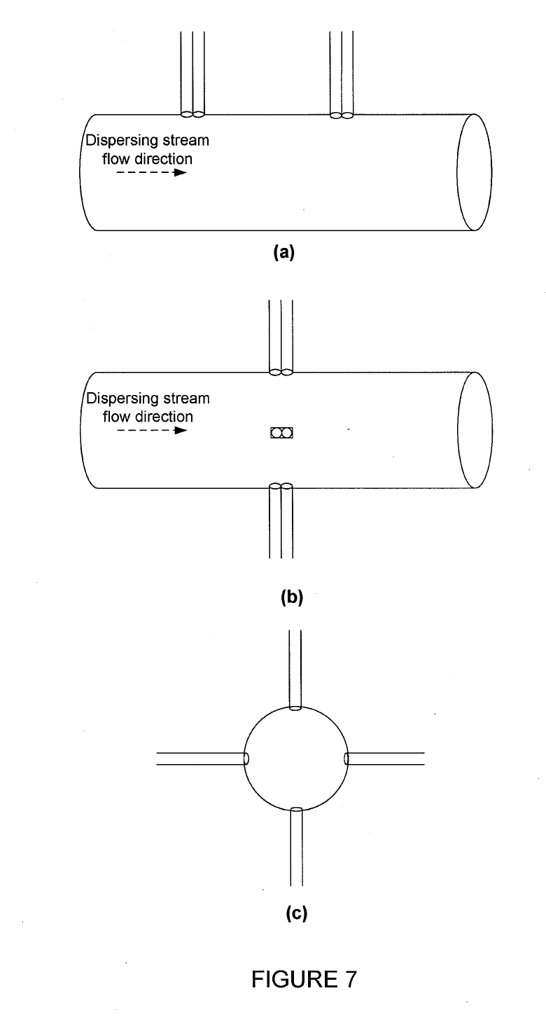

FIG. 8a-b illustrates an embodiment of the method of making a nanoparticles. FIG. 8a is a perspective view of a system for making nanoparticles. Liquid feed channel 800 outputs into dispersing channel 810. Liquid feed stream 820 flows through liquid feed channel 800 and exits to contact dispersing stream 830. Liquid feed stream 820 solidifies to form nanoparticle 840 after contacting dispersing stream 830. Liquid feed stream 820 and dispersing stream 830 may, in some embodiments, continuously flow, such that a plurality of nanoparticles form in the dispersing stream.

Applicants have found that, in some embodiments, small nanoparticles can be achieved without using a correspondingly small liquid feed stream. Thus, some embodiments of the method include forming nanoparticles having a diameter that is a small fraction of the diameter of the liquid feed stream.

The precise conditions for obtaining a certain nanoparticle size may be empirically determined in view of the guidance provides herein, including examples of suitable conditions, as well as various factors that affect nanoparticle size. A desired nanoparticle size may be achieved by adjusting at least three factors: (i) the size (e.g., diameter) of the liquid feed stream; (ii) the Reynolds number (Re) for the dispersing stream; and (iii) the Plateau-Rayleigh instability for the liquid feed stream, i.e., the propensity of the liquid to adopt a morphology that minimizes surface area.

First, the size of the liquid feed stream can affect the size of the nanoparticles. For example, by decreasing the diameter of a liquid feed stream, the nanoparticles will be smaller. Without being bound to any particular theory, it is believed the size of the liquid feed stream limits the size of the initial liquid droplets that solidify into the nanoparticles.

Although the size of the liquid feed stream can be adjusted to change the size of the nanoparticles, it may also be possible to have a relatively large liquid feed stream and still obtain small nanoparticles. This can be achieved by varying other parameters, such as the Reynolds Number of the dispersing stream, to shear off the droplets from the feed stream, and thereby decrease the size of the resultant particles.

The Reynolds Number (Re) can be defined as: Re=ρVL/μ, where: ρ is the density of the fluid (kg/m³); V is the mean fluid velocity (SI units: m/s); L is a characteristic length (traveled length of fluid) (m); and μ is the dynamic viscosity of the fluid (Pa·s or N·s/m² or kg/m·s)

As one example of how the Reynolds Number affect nanoparticle size, we have found that increasing the dispersing stream flow rate—which in turn increases the Reynolds Number—will decrease the diameter of PLGA nanoparticles. As another example, we have found that decreasing the dynamic viscosity (μ) of the dispersing stream will decrease the diameter of PLGA nanoparticles. The viscosity was decreased by adding methanol to the aqueous dispersion stream. Of course, other solvents can be selected to vary the dynamic viscosity and in turn adjust the size of the nanoparticles.

Third, the Plateau-Rayleigh instability will also affect the size of the nanoparticles. The Plateau-Rayleigh instability can be modified by the various materials included in the liquid feed stream. Non-limiting examples of materials in the liquid feed stream that can affect the nanoparticle size include: the solvent(s), optional surfactant(s), and the solidifying component(s) that form the nanoparticles (e.g., a polymer, such as PLGA).

As an example, decreasing the concentration of PLGA in the liquid feed stream will also decrease the size of the nanoparticles. Accordingly, a person of ordinary skill, guided by the teachings of the present application, can select appropriate combinations of materials to adjust the nanoparticle size.

The systems and methods disclosed herein may therefore provide advantages over existing microfluidic devices used for preparing nanoparticles. Microfluidic platforms generally utilize very small (μm in diameter) flow channels (e.g., an about 20 μm by 60 μm channel), which in turn, constrains the initial size of the droplets that are formed. However, the small channels in the microfluidic systems prevent high flow rates. Microfluidic channels generally cannot accommodate flow rates greater than ˜100 μL/min because the increased pressure usually causes breaks or leaking.

In contrast, the systems and methods disclosed herein can provide a solution to the problems of microfluidic channels by using a dispersing channel with a larger diameter. This allows the use of a higher mean fluid velocity and a corresponding increase in Reynolds Number. Therefore, increasing the size of the dispersing channel allows for higher fluid velocities and enables the formation of smaller nanoparticles.

The dispersing stream may, for example, have a diameter greater than about 500 μm. In some embodiments, the dispersing stream has a diameter of at least about 1 mm. In some embodiments, the dispersing stream has a diameter of at least about 2 mm. In some embodiments, the dispersing stream has a diameter of at least about 5 mm. In some embodiments, the dispersing stream has a diameter of at least about 10 mm.

As discussed above, the flow rate in the dispersing channel may vary according to the desired size of the nanoparticles. However, the flow rate is not particularly limited. For example, the flow rate in the dispersing stream may be as much as 100 mL/min. or more. Meanwhile, the flow rate in the dispersing stream may be as little as 1 mL/min or less.

In some embodiments, the flow rate in the dispersing stream is at least 10 mL/min. As an example, the flow rate in the dispersing stream may be at least about 20 mL/min; at least about 40 mL/min; or at least about 50 mL/min. In some embodiments, the flow rate in the dispersing stream may be no more than about 200 mL/min. As an example, the flow rate may be no more than about 100 mL/min; no more than about 80 mL/min; or no more than about 60 mL/min.

Also, the size of the liquid feed stream is not particularly limited, and may be adjusted to change the size of the nanoparticles. The liquid feed stream can, in some embodiments, have a diameter in the range of about 1 μm to about 1 mm. As an example, the liquid feed stream can be at least about 1 μm; at least about 10 μm; at least about 50 μm; at least about 100 μm; at least about 250 μm; or at least about 500 μm. Furthermore, the liquid feed stream can be no more than about 1 mm; no more than about 750 μm; no more than about 500 μm; no more than about 250 μm; or no more than about 100 μm. In some embodiments, the liquid feed stream has a diameter greater than about 1 mm.

The flow rate of the liquid feed stream can vary, but may generally be in the range of about 1 μL/hr to about 100 mL/min. The flow rate of the liquid feed stream may, for example, be at least about 0.5 μL/min; at least about 1 μL/min.; at least about 2 μL/min; or at least about 3 μL/min. The flow rate of the liquid feed stream may also be, for example, no more than about 10 mL/min; no more than about 1 mL/min; no more than about 100 μL/min; or no more than about 10 μL/min.

Generally, the contents of the liquid feed stream may be selected based upon the desired properties of the nanoparticles. And the components may, for example, be any of those disclosed above with respect to Janus particles. The liquid feed stream can include, for example, one or more solidifying components dispersed in a solvent. In some embodiments, the solidifying components include a polymer. The amount of solidifying components in the liquid feed stream is not particularly limited, but may be, for example, in the range of about 1 mg/mL and 100 mg/mL. As an example, the amount of solidifying components in the liquid feed stream can be at least about 10 mg/mL; at least about 20 mg/mL; at least about 40 mg/mL; or at least about 50 mg/mL. Also, the amount of solidifying components in the liquid feed stream can be no more than about 80 mg/mL; no more than about 60 mg/mL; or no more than about 40 mg/mL.

In some embodiments, the liquid feed stream is an emulsion. An emulsion may be desired when solidifying a mixture of hydrophobic and hydrophilic components into a single nanoparticle. For example, nanoparticles having a mixture of a hydrophilic drug and a hydrophobic polymer may be prepared using an emulsion in the liquid feed stream. The emulsion may be a stable emulsion or an unstable emulsion. Moreover, the emulsion may be prepared using standard techniques for intermixing the components, such as stirring, sonicating, high shear blending, and the like. It is preferred that the emulsion is well-mixed prior to contacting the dispersing stream to obtain a generally uniform dispersion of components in the nanoparticle.

Generally, an emulsion can include a first solvent, a second solvent, and one or more solidifying components, where there two solvents are immiscible, or at least partially immiscible. In some embodiments, the emulsion includes water, an organic solvent (e.g., chloroform, dichloromethane, ethyl acetate, etc.), and a polymer (e.g., PLGA).

The emulsion may also optionally include one or more surfactants. The surfactant is not particularly limited and may be selected based on the desired properties of the emulsion. The surfactant can be, for example, an ionic surfactant (e.g., sodium dodecylsulfate), a zwitterionic surfactant (e.g., dodecyl betaine), or a non-ionic surfactant (e.g., poloxamer).

Although the shape of the nanoparticles is not particularly limited, the nanoparticles can, for example, be generally spherical. In some embodiments, the nanoparticles are not hollow. In some embodiments, the nanoparticles are substantially symmetric. The nanoparticles may optionally include a pharmaceutical agent, such as those discussed above with respect to Janus particles. For example, the nanoparticles may include an anti-cancer drug, such as paclitaxel or doxorubicin.

In some embodiments, at least a portion (e.g., at least 20%, at least 50%, at least 80%, at least 90%, or at least 95%) of the plurality of nanoparticles can have a first diameter in the nanometer-range. The first diameter can be, for example, at least about 10 nm; at least about 20 nm; at least about 50 nm; at least about 100 nm; or at least about 150 nm. Furthermore, the first diameter of the nanoparticles can be, for example, no more than about 1000 nm; no more than about 500 nm; no more than about 300 nm; or no more than about 200 nm. These ranges may, in some embodiments, be obtained without removing nanoparticles within certain diameter ranges (e.g., filtering).

The methods disclosed herein may also, in some embodiments, produce a plurality of nanoparticles having a small size distribution. The size distribution may, in some embodiments, be obtained without removing nanoparticles within certain diameter ranges (e.g., filtering). In some embodiments, the method produces a plurality of nanoparticles (e.g., at least about 100 nanoparticles, at least about 1000 nanoparticles, etc.) that have a low standard deviation from the average diameter. For example, the standard deviation may be no more than about 25% of the average diameter. In some embodiments, the standard deviation may be no more than about 20% of the average diameter. In some embodiments, the standard deviation may be no more than about 15% of the average diameter. In some embodiments, the standard deviation may be no more than about 10% of the average diameter.

The diameter of the nanoparticles can optionally be a small fraction of the diameter of the liquid feed stream. For example, the liquid feed stream may have a diameter of about 110 μm and yield nanoparticles with a diameter about 110 nm. Therefore, the nanoparticle diameter is about 1/1000 of the diameter of the liquid feed stream in this example. In some embodiments, the diameter of the nanoparticle is no more than about 1/200 of the diameter of the liquid feed stream. In some embodiments, the diameter of the nanoparticle is no more than about 1/400 of the diameter of the liquid feed stream. In some embodiments, the diameter of the nanoparticle is no more than about 1/500 of the diameter of the liquid feed stream. In some embodiments, the diameter of the nanoparticle is no more than about 1/750 of the diameter of the liquid feed stream.

Although very small nanoparticles can be formed according to the teachings of the present application, it is also possible to produce larger particles by adjusting the various factors discussed above. For example, the larger particles may be formed by increasing the polymer concentration in the liquid feed stream, or decreasing the flow rate of the dispersing stream. In some embodiments, the method can form particles that have a diameter ranging from about 1 μm to about 1 mm. The diameter of the particles can be, for example, at least about 1 μm; at least about 10 μm; at least about 50 μm; at least about 100 μm; or at least about 200 μm. Moreover, the diameter of the particles can be, for example, no more than about 1000 μm; no more than about 750 μm; no more than about 500 μm; or no more than about 200 μm.

The method and systems disclosed herein may advantageously provide a high yield of nanoparticles from the liquid feed stream. That is, the weight of nanoparticles formed is a large portion of the total weight of solidifying material contacting the dispersing stream. For example, a liquid feed stream may have 5 grams of PLGA dispersed in a solvent. If the entire amount of the liquid feed stream contacts the dispersing stream to form 4 grams of nanoparticles, the yield is 80%. The method and systems disclosed herein can, for example, exhibit yields of at least about 25%; at least about 50%; at least about 75%; at least about 80%; or at least about 90%.

### EXAMPLES

Additional embodiments are disclosed in further detail in the following examples, which are not in any way intended to limit the scope of the claims.

### Example 1

Janus particles having two components, each with different forms of poly(lactic-co-glycolic acid) (PLGA) and containing distinct fluorophores were prepared using a system generally configured as illustrated in FIG. 3. One liquid feed stream contained a solution of 25 mg/mL of PLGA 7502 (75/25, Inherent Viscosity of 0.19 g/mL) in dimethylformamide (DMF) and Nile red. A second liquid feed stream contained a solution of 25 mg/mL PLGA (Resomer RG504H, Inherent Viscosity of 0.54 g/mL) in acetone and rhodamine-6G. Both liquid feed streams were fed through separates 26 s stainless steel needles (inner diameter of about 0.11 mm). TYGON tubing (ID 3/32′, OD 5/32′) form the dispersing channel and contained a solution of 1% polyvinyl alcohol in water. The flow rate of both the liquid feed streams was set at 1.6 μL/min, while the dispersing channel was at 10 mL/min.

The morphology of the particles was analyzed by confocal laser scanning microscopy and showed particles with distinct fluorescence on opposite sides, which was attributed to the two different fluorophores in the liquid feed streams. Atomic force microscopy revealed the particles have an average diameter of ˜200 nm. Meanwhile, dynamic light scattering confirmed the homogeneity of the population, where greater than 99% of the particles had a diameter of 199±31 nm.

### Example 2

Janus particles were prepared from two polymer solutions: (i) Solution A containing paclitaxel, and (ii) Solution B containing doxorubicin. Solution A was prepared by dissolving 1 mg paclitaxel and 25 mg PLGA (PG5002, 50/50 monomer ratio, inherent viscosity of about 0.2 dl/g) in 1 ml acetonitrile. Solution B was prepared by first dissolving 1 mg doxorubicin in 1.5 mL of 1% PVA solution and the resulting solution was added directly to a PLGA (RESOMER 502H, 50/50 monomer ratio with charged end groups, inherent viscosity of about 0.16 to 0.24 dl/g) solution of 50 mg polymer in 1.5 mL methylene chloride/methanol (2:1). This solution was sonicated on ice for 60 seconds to form a doxorubicin-containing emulsion. 1 mL of each sample solution was injected at a flow rate of 200 μL/hour into a 40 mL dispersing phase (1% PVA solution, 75 mL/min) through a 26 s needle (inner diameter of about 0.11 mm). Janus nanoparticles were collected into a beaker containing the same solution. Janus particles were washed 3 times by Millipore water and lyophilized before use.

Paclitaxel content in the Janus particles was assayed by reverse phase HPLC. Briefly, 1 mg of particles was dissolved in 1 ml acetonitrile under vigorous vortexing. This solution was centrifuged and a clear solution was obtained for HPLC analysis. The mobile phase of HPLC was composed of equal parts acetonitrile and water (v/v). The concentration of paclitaxel in the Janus particles was obtained by calculating from a standard curve. The encapsulation efficiency was calculated as the mass ratio of the entrapped drug in nanoparticles to the amount used in their preparation.

The doxorubicin concentration in the Janus particles was assayed using a Molecular Devices SPECTRAMAX GEMINI EM microplate reader. Briefly, 1 mg of particles was dissolved in 1 mL DMSO under vigorous vortexing. The fluorescence of the solution was measured at excitation 480 nm/emission 590 nm and compared with a standard curve to determine the doxorubicin concentration. Encapsulation efficiency was calculated as the mass ratio of the entrapped drug in the Janus particles to the amount used in their preparation.

The Janus particles contained 0.6% doxorubicin, with an encapsulation efficiency of 15%. The Janus particles contained 1.15% paclitaxel, with an encapsulation efficiency of 80%.

The drug delivery profile for the Janus particles was determined as a function of time during incubation in 1× PBS containing 0.1% tween 80. 1 mg samples of Janus particles were suspended in 1 mL PBS in a microcentrifuge tube and sonicated briefly in an ultrasonic water bath. The samples were then incubated on an orbital shaker at 37° C. The Janus particles were centrifuged at 13.1K rpm for 30 minutes and supernatant removed and replaced with fresh solution at defined time points. The supernatant was lyophilized and the drug extracted using acetonitrile (for paclitaxel) or DMSO (for doxorubicin) and the concentration was determined using the same methods described above.

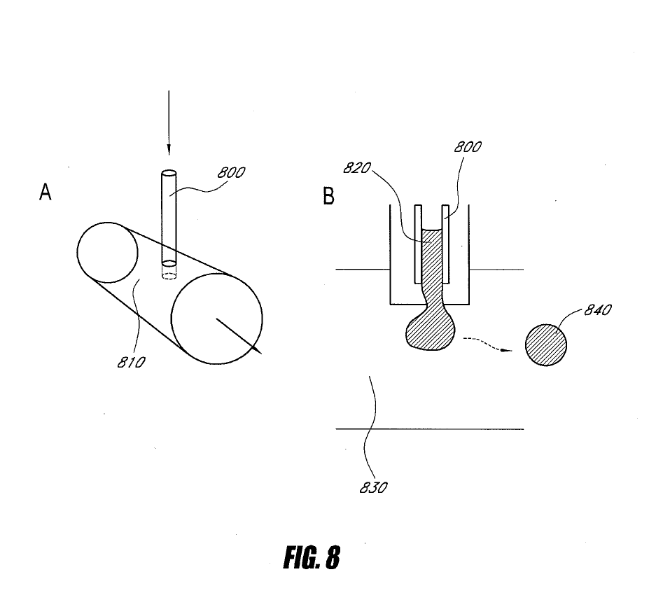

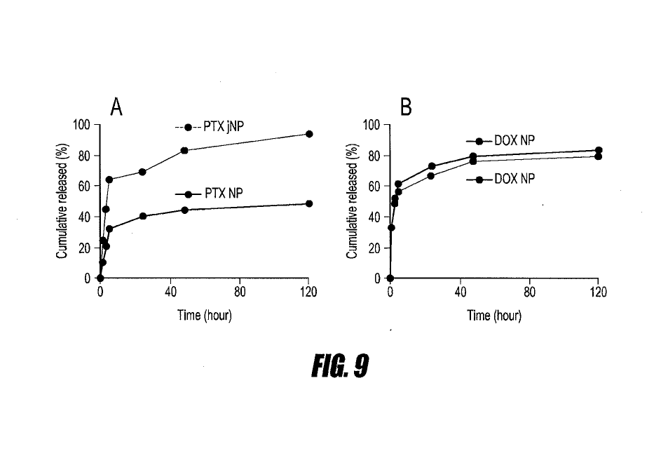

The drug delivery profile for paclitaxel in the Janus particles is shown in FIG. 9a (dashed line). The drug delivery profile for doxorubicin in the Janus particles is shown in FIG. 9b (dashed line). Both drugs exhibit an initial burst of drug release within the first 2 hours. Subsequently, a slower, sustained release occurs for both drugs.

### Example 3

Paclitaxel or doxorubicin containing PLGA nanoparticles were prepared by injecting 1 mL of Solution A or Solution B (as described above in Example 2) using a 26 s needle at 200 μL/hour into a 40 mL dispersing phase (1% PVA solution, 75 mL/min). Nanoparticles were collected into a beaker containing the same solution. Particles were washed 3 times by Millipore water and lyophilized before use.

The paclitaxel- and doxorubicin-containing nanoparticles were each separately analyzed using the same techniques described in Example 2.

The nanoparticles loaded with paclitaxel contained 3.44% paclitaxel (w/w), with an encapsulation efficiency of 86%. Nanoparticles loaded with doxorubicin contained 1.25% doxorubicin (w/w), with an encapsulation efficiency of 19%.

The drug delivery profile for the paclitaxel-containing nanoparticles is shown in FIG. 9a (solid line). The drug delivery profile for the doxorubicin-containing nanoparticles is shown in FIG. 9b (solid line). The nanoparticles also exhibited an initial burst of drug release within the first 2 hours. Subsequently, a slower sustained release occurred for both types of nanoparticles. Interestingly, the drug delivery profile for doxorubicin in the nanoparticle was similar to the Janus particles (i.e., Example 2). Meanwhile, the nanoparticles released more paclitaxel after 120 hours compared to the Janus particles.

### Example 4

The liquid feed stream was prepared by dissolving 20 mg/mL PLGA (RESOMER RG502H, Boehringer-Ingelheim) in acetonitrile. The resulting PLGA solution was injected through a 26 s stainless steel needle (inner diameter of about 0.11 mm) into a TYGON tubing (ID 3/32′, OD 5/32′) that was used to pass the dispersing phase. The needle was inserted to the interior at 50% of the tubing diameter. The PLGA solution fed into the dispersing channel with a 3 ml syringe controlled by a single syringe pump (KDS100, KD Scientific, Massachusetts, USA). A stream of surfactant (1% PVA solution, 20 ml) passing through the dispersing channel (Tygon® tubing with ID 3/32′, and OD 5/32′) was controlled by a Fisher Scientific Variable-Flow Peristaltic Pump.

Liquid feed stream samples (about 0.2 ml) were injected at a flow rate of 3.2 μl/min into the dispersing stream. The dispersing stream had a flow rate of 35 mL/min. Nanoparticles were collected into a beaker for analysis. The nanoparticles were washed by centrifuging for 15 minutes using an Eppendorf 5415R at 13200 rpm at room temperature and then removing the supernatant. The nanoparticles were resuspended in DI water by bath sonication (Branson's Model B200). This was repeated three times and the final suspension was sent for analysis.

SEM experiments were conducted by depositing the nanoparticle suspension on freshly cleaved mica and allowing them to dry. A thin film of Au was sputtered onto these mica substrates with sample. Samples were imaged with scanning electron microscopy (SEM; JEOL 5800LV) without filtration or purification. Particle size was measured by using ImageJ. For each sample, the mean diameter was calculated based on the measurements of 100 randomly chosen particles.

The nanoparticles exhibited an average diameter of 327±19 nm.

### Examples 5 and 6

Nanoparticles were prepared and analyzed according to generally the same methods disclosed in Example 4 except that the PLGA concentration was 10 mg/mL or 40 mg/mL. The 10 mg/mL liquid feed stream produced nanoparticles with an average diameter of 231±35 nm. The 40 mg/mL liquid feed stream produced nanoparticles with an average diameter of 393±38 nm.

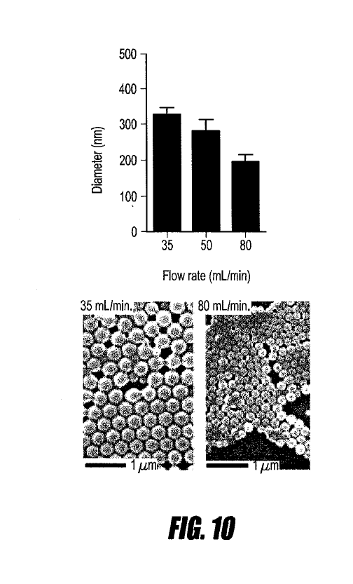

The results from Examples 4-6 are shown in FIG. 10.

### Examples 7 and 8

Nanoparticles were prepared and analyzed according to generally the same methods disclosed in Example 4 except that the dispersing stream flow rate was 50 mL/min or 80 mL/min. The 50 mL/min dispersing stream produced nanoparticles with an average diameter of 278±35 nm. The 80 mL/min dispersing stream produced nanoparticles with an average diameter of 193±19 nm.

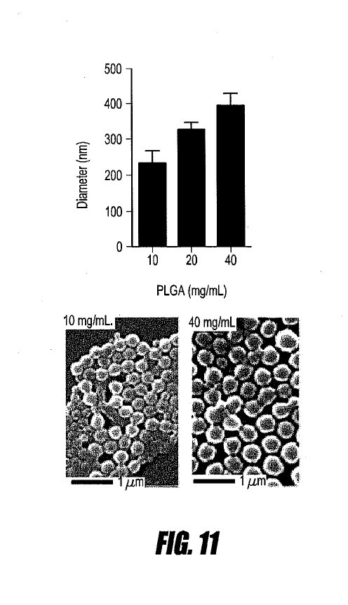

The results from Examples 4, 7, and 8 are shown in FIG. 11.

### Examples 9-11

Nanoparticles were prepared and analyzed according to generally the same methods disclosed in Example 4 except that the dispersing stream flow rate was 50 mL/min and the dispersing stream included 20%, 50%, or 80% methanol (v/v). The 20% methanol dispersing stream produced nanoparticles with an average diameter of 512±45 nm. The 50% methanol dispersing stream produced nanoparticles with an average diameter of 315±36 nm. The 80% methanol dispersing stream produced nanoparticles with an average diameter of 148±14 nm.

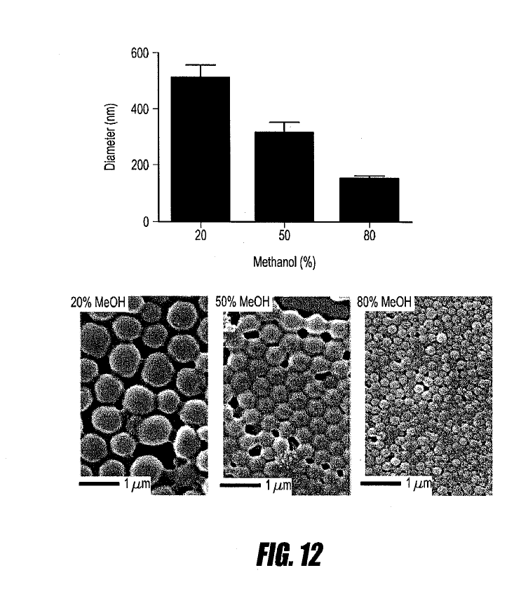

The results from Examples 9-11 are shown in FIG. 12.

### Comparative Example 1

PLGA nanoparticles were prepared using the same polymer and solvents systems as Example 4; however, a microfluidic device was used similar to those described in, for example, Karnik R, et al., Microfluidic platform for controlled synthesis of polymeric nanoparticles., Nano Lett. 8:2906-2912 (2008), the contents of which are hereby incorporated by reference in its entirety. The nanoparticles were analyzed using generally the same methods as described in Example 4 and exhibited an average diameter of 211±70 nm.

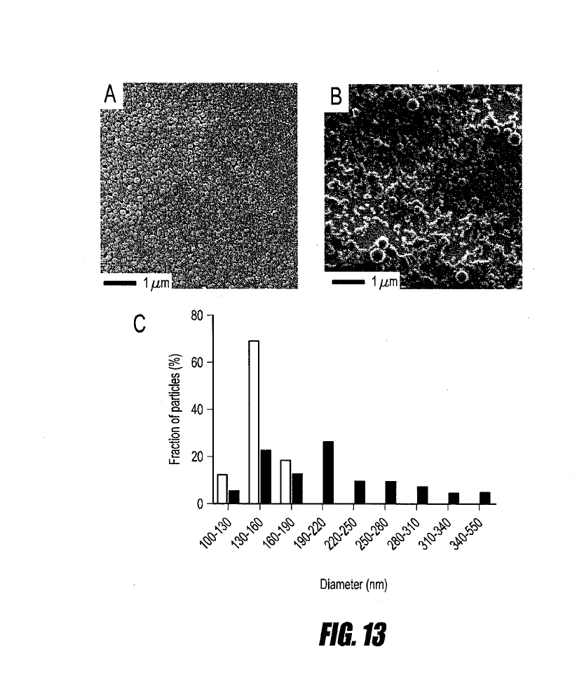

FIG. 13 compares the nanoparticles formed according to Example 11 (80% methanol dispersing stream) and Comparative Example 1. FIG. 13a is an SEM image of the nanoparticles in Example 11, while FIG. 13b is an SEM image of the nanoparticles in Comparative Example 1. FIG. 13c shows the size distribution of nanoparticles for Example 11 (white bars) and Comparative Example 1 (black bars).

1. A method of making Janus particles, comprising: (a) providing at least a first liquid feed stream and a second liquid feed stream; and (b) intermixing the first liquid feed stream and the second liquid feed stream with a dispersing stream, thereby solidifying components of the first liquid feed stream and the second liquid feed stream into a plurality of Janus particles dispersed in the dispersing stream, wherein: the first liquid feed stream comprises a first polymer and the second liquid feed stream comprises a second component that is substantially different from the first polymer; and at least a portion of the Janus particles comprise the first polymer and the second component.

2. The method of claim 1, wherein a portion of the first liquid feed stream contacts a portion of the second liquid feed stream before the portion of the first liquid feed stream and/or the portion of the second liquid feed stream contacts the dispersing stream.

3. The method of claim 1, wherein a portion of the first liquid feed stream, a portion of the second liquid feed stream and the dispersing stream all initially contact each other at about the time.

4. The method of claim 1, wherein a portion of the first liquid feed stream and/or a portion of the second liquid feed stream contacts the dispersing stream before the portion of the first liquid feed stream contacts the portion of the second liquid feed stream.

5. The method of claim 1, wherein the first liquid feed stream further comprises a first solvent that is at least partially miscible in the dispersing stream.

6. The method of claims 5, wherein the first liquid feed stream further comprises a first solvent selected from the group consisting of 1,4 dioxane, tetrahydrofuran (THF), acetone, acetonitrile, dimethyl sulfoxide (DMSO), dimethylformamide (DMF), acids, and C₁-C₈ alcohols.

7. The method of claim 6, wherein the second liquid feed stream further comprises a second solvent that is at least partially miscible in the dispersing stream.

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. A composition comprising a plurality of Janus particles, each Janus particle comprising: a first component comprising a first polymer; and a second component that is substantially different from the first component, wherein: the Janus particles have an average size in the range of about 10 nm to about 2000 nm; and at least part of the first component and at least part of the second component are exposed at an outer surface of the Janus particle.

27. A system for making a plurality of Janus particles, comprising: a first feed channel; a second feed channel; and a dispersing channel, wherein: the first feed channel has a first outlet that is operably connected to the dispersing channel; the second feed channel has a second outlet that is operably connected to the dispersing channel; the first outlet and the second outlet are no more than about 5 mm apart; and the first outlet and the second outlet are within about 1 mm of the dispersing channel; and the first feed channel has a first diameter in the range of about 10 μm to about 1 mm; the second feed channel has a second diameter in the range of about 10 μm to about 1 mm; and the dispersing channel has a third diameter that is at least 2 times larger than the first diameter.

28. (canceled)

29. (canceled)

30. The system of claim 27, further comprising one or more pumps configured to displace a substance in the first feed channel, the second feed channel and/or the dispersing channel.

31. The system of claim 27, further comprising a means for isolating Janus particles dispersed in the dispersing channel, the isolating means being operably connected to the dispersing channel.

32. The system of claim 31, wherein the isolating means comprises a filter.

33. The system of claim 27, wherein the dispersing channel forms a closed loop.

34. The system of claim 27, further comprising a processor in communication with one or more pumps and/or one or more measuring devices.

35. The system of claim 27, further comprising one or more additional feed channels connected to the dispersion channel at a common intersection with any other feed channel.

36. The system of claim 27, wherein the first outlet and the second outlet are operably connected to a cojoining chamber that is operably connected to the dispersing channel.

37. The system of claim 36, wherein the cojoining chamber is configured so that the first outlet or the second outlet is at least about 10 nm from the dispersing channel.

38. The system of claim 36, wherein the cojoining chamber is configured so that at least one of the first outlet and the second outlet is no more than about 100 μm from the dispersing channel.

39. The system of claim 27, where at least one of the first feed channel, the second feed channel and the dispersing channel is prepared by lithography, embossing, or molding of a polymer.

40. The system of claim 27, where at least one of the first feed channel, the second feed channel and the dispersing channel is a plastic tubing or a stainless steel tubing.
